(19) United States
(12) Patent Application Publication
(10) Pub. No.: US 2026/0263376 A1
(43) Pub. Date: Sep. 10, 2026

(54) THERAPEUTIC DELIVERY VECTORS COMPRISING AMPHIPHILIC BLOCK COPOLYMERS

(71) Applicant: Swansea University, Swansea (GB)

(72) Inventors: Kadie EDWARDS, Swansea (GB); Lewis FRANCIS, Swansea (GB)

(21) Appl. No.: 19/165,821

(22) PCT Filed: Mar. 18, 2024
(86) PCT No.: PCT/GB2024/050727

§ 371 Date: Sep. 16, 2025

(30) Foreign Application Priority Data
Mar. 17, 2023 (GB) 2303956.3

(51) Int. Cl. A61K 9/51; A61K 33/243; A61K 45/06; A61P 35/00

(52) U.S. Cl. CPC A61K 9/5153; A61K 9/5192; A61K 33/243; A61K 45/06; A61P 35/00

(57) ABSTRACT

A nanoparticle for encapsulation of one or more active pharmaceutical ingredient, said nanoparticle comprising an amphiphilic block copolymer, wherein said amphiphilic block copolymer comprises: i. a hydrophobic polymer block comprising structural units of trimethylene carbonate; and ii. a hydrophilic polymer block comprising structural units of one or more alkylene glycols. A drug delivery vector comprising one or more active pharmaceutical ingredients encapsulated within said nanoparticle. A process for preparing said nanoparticle, the process comprising: i. dissolving said amphiphilic block copolymer in an oligomeric alkylene glycol solution to prepare an alkylene glycol-polymer solution; and ii. hydrating said alkylene glycol-polymer solution by addition of water, an aqueous solution or an aqueous buffer to prepare said nanoparticle. A process for preparing said drug delivery vector by encapsulating one or more active pharmaceutical ingredients within a nanoparticle, wherein said process comprises: i. co-dissolving said amphiphilic block copolymer and said one or more active pharmaceutical ingredients in an oligomeric alkylene glycol solution to prepare an alkylene glycol-polymer solution; and ii. hydrating said alkylene glycol-polymer solution by the addition of water, an aqueous solution or an aqueous buffer to prepare said drug delivery vector. Said drug delivery vector for use as a medicament, preferably for use in the treatment of cancer.

### FIELD OF THE INVENTION

The invention relates to a nanoparticle that is suitable for use as a nanoscopic vector for delivering therapeutic agents, and to methods for the manufacture of said nanoparticles and/or vectors; a pharmaceutical or veterinary composition comprising said vectors; the use of said vectors as a medicament; and the use of said vectors for treatment of cancer. The invention further relates to a method of treatment employing the use of said vectors or pharmaceutical compositions.

### BACKGROUND OF THE INVENTION

The vast majority of cancers currently have unmet clinical needs, with a major mitigating factor to treatment being the need to administer tolerated doses (TD) opposed to optimal doses (OD) as systemic exposure of chemotherapeutics cause catastrophic side effects in patients. Whilst many solid tumour cancers are undermanaged there has been an overall improvement in combined 10-year cancer survival rates in Wales and England from 24.0% in 1971 to 49.8% in 2011. However, this is not reflected in ovarian cancer (OC) prognoses. Recent reviews have highlighted that the 5-year survival rates have stagnated since 1980's and OC 10-year survival rates are approximately 35.3% based on CRUK data, averaged over all OC grades and subtypes. This puts OC survival rates lower than the general average for all cancers and highlights the need for improved therapeutic regimens.

Additionally, OC presents with unique clinical difficulties, namely the frequent metastasis through the peritoneal cavity into the omentum, fast refectory rates of disease, where treatments become ineffective following resistance and vast heterogeneity in the disease. Intraperitoneal (IP) delivery of therapeutics is a promising option to improve drug exposure to the neoplasm whilst simultaneously decreasing the systemic exposure, potentially reducing side-effects. However, current IP regimes use therapeutics designed for IV administration, with little consideration of optimization for this route of administration, with pharmacokinetic studies of IP administered docetaxel or paclitaxel showing clearance in under 24 h for example. There is a clear need for improved therapeutics that are compatible with IP delivery, and address the many challenges present in this disease, helping to alleviate the present clinical burden.

Targeted drug delivery has been the focus of intensive medical research, particularly in relation to the administration of harmful active pharmaceuticals agents such as chemotherapeutics. Such systems are designed to deliver medication to a patient in a manner that increases the concentration in some parts of the body relative to others. This means of delivery is largely founded on nanomedicine, which plans to employ nanoparticle-mediated drug delivery in order to combat the downfalls of conventional drug delivery.

Research over the years has contributed numerous advanced drug delivery systems, mostly comprising nanoparticulate carriers, to the list of pharmaceutical products. Some of these include nanoparticles—polymeric and lipidic, liposomes, dendrimers, micelles, nanoemulsions and nanosuspensions. Their rapid development arises from their ability to overcome the drawbacks of the currently employed therapeutic drugs, which exhibit poor biopharmaceutical and pharmacokinetic properties. However, the majority of these are insoluble or possess poor aqueous solubility, thus presenting formulation challenges, since solubility is critical for determining the drug efficacy irrespective of the administration route.

The goal of a targeted drug delivery system is to prolong, localize, target and have a protected drug interaction with target tissue. The conventional drug delivery system is the absorption of the drug across a biological membrane, whereas the targeted release system releases the drug in a dosage form. An ideal drug delivery vehicle must be non-toxic, biocompatible, non-immunogenic, biodegradable, and must avoid recognition by the host's defence mechanisms. However, the ubiquity of harmful solvents and complex methodologies in the fabrication of drug-loaded delivery vectors for cancer therapeutics is rarely brought into contention due to the lack of alternative strategies. In the development of therapeutic nanoscopic vectors there are a number of issues that typically cause contention: (1) the use of harmful substances in the fabrication process, (2) elucidating the exact physicochemical nature of the resulting nanostructure and (3) identifying the key physical features that determine biochemical performance. Addressing these issues is especially pertinent because so many nanomedical formulations fail to reach the clinic due to unforeseen complications.

Therefore, the problems of carrier toxicity, specificity and producibility create a bottleneck in the development of new nanomedical technologies.

The implementation of polymers as drug delivery systems is an exciting method for IP compatible drug formulations due to the aptitude to fine-tune physiochemical properties of both the bulk material as well as the nanocarrier, the compatibility with a vast array of payloads and choice of passive or covalent encapsulation. Ultimately, these factors allow for custom formulations that could be suitable for a range of diseases. Successful polymer formulations range from relatively simple polymer-drug conjugates, regularly used to improve pharmacokinetics such as circulation time, to more advanced polymer systems such as polymer nanoparticles (NPs), micelles and polymersomes. However, unfortunately there is clear disconnect between in vitro development and performance in vivo, formulations show great promise at an early stage, but ultimately many fail to impress and proceed to clinic. There is, therefore, an unquestionable need for additional, effective NP drug delivery systems.

Poly(ethylene glycol)-block-poly(trimethylene carbonate) (PEG-PTMC) is a remarkably versatile polymer with straightforward chemistry that may be tailored for a vast array of applications. Direct hydration of this polymer has been used to form small drug delivery vectors for delivering active pharmaceutical ingredients (e.g. parthenolide (PTL)) in the treatment of liquid cancers such as Acute lymphoblastic leukaemia (ALL), without the use of harsh chemicals or the need for additional purification steps (Ridolfo et al. Small 2018, 14, 1703774). Avoiding a multi-step process prevents complications such as loss of starting material, including any given drug or payload and vastly decreases fabrication time by 30-432× compared to other polymer NPs. However, delivering active therapeutic agents deep within solid tumour cancers presents a fundamentally different challenge to the delivery and treatment of liquid cancers.

The present application addresses the need to provide additional, effective NPs that can be used as drug delivery systems, particularly for the treatment of solid tumour cancers. Further, the invention may provide a novel drug delivery system that is optimised for intraperitoneal and/or intravenous administration.

Further still, use of the NPs of the invention as drug delivery systems for the treatment of solid tumour cancers, in particular ovarian cancer, may improve prognosis compared with conventional treatment regimens.

As a proof of principle study, using direct hydration, we employed molecular engineering to demonstrate control over the size and composition of PEG-PTMC nanoparticles, which possess exceptional size reproducibility (dispersity), which is vital in a drug delivery platform. Drug (cisplatin) delivery vectors were prepared by drug-loading of nanoparticles, yielding stable loadings of up to 20 wt %. Significantly, cisplatin loaded vectors were fabricated without need for further purification due to complete drug encapsulation. Excellent physical stability and drug release characteristics were observed. Moreover, our findings suggested that the cisplatin nanovectors, particularly when administered via intravenous or intraperitoneal route, display a greater reduction in tumour volume in ovarian cancer cell as compared to the free drug.

The platform therefore represents a facile methodology with the potential to enhance producibility and improve therapeutic performance by reducing carrier toxicity and facilitating effective drug delivery. The ease of formulation and absence of toxic components makes this an extremely attractive candidate for development towards drug delivery applications.

### STATEMENTS OF INVENTION

The present invention, in its various aspects, is as set out in the accompanying claims.

According to a first aspect, the invention provides a nanoparticle for encapsulation of one or more active pharmaceutical ingredients, said nanoparticle comprising an amphiphilic block copolymer, wherein said amphiphilic block copolymer comprises:
i. a hydrophobic polymer block comprising structural units of trimethylene carbonate; and
ii. a hydrophilic polymer block comprising structural units of one or more alkylene glycols.

Reference herein to a nanoparticle is to an aggregate structure composed of amphiphilic block copolymer molecules arranged such that the inner core of the aggregate structure comprises hydrophobic subunits, and the outer surface comprises hydrophilic subunits, of the amphiphilic block copolymer molecules. In the context of this application, the term nanoparticle includes within its scope micelles (i.e. closed aggregate structures in which the core of said structure is a hydrophobic environment formed from the hydrophobic subunits of amphiphilic block copolymer molecules) and polymersomes (i.e. closed aggregate structures composed of a plurality of layers formed from amphiphilic block copolymer molecules, wherein the polymer molecules encapsulate an internal aqueous core. Preferably, the term nanoparticle refers to a micelle.

Preferably, said nanoparticles have an average particle size, measured according to dynamic light scattering (DLS), from about 50 to 500 nm, more preferably from about 75 nm to about 250 nm, and most preferably from about 85 nm to about 150 nm.

Reference herein to an amphiphilic block copolymer is to a copolymer comprising one or more hydrophobic polymer subunits covalently linked to one or more hydrophilic polymer subunits. Preferably each of the hydrophobic and hydrophilic polymer subunits are homopolymer subunits. Preferably the block copolymer is a diblock copolymer, i.e. a block copolymer comprising a single hydrophobic polymer subunit covalently linked to a single hydrophilic polymer subunit.

The hydrophobic block of the amphiphilic block copolymer comprises structural units of trimethylene carbonate. Preferably, the hydrophobic block comprises, based on the total weight of the structural units used to form said hydrophobic block, no more than 10 weight percent, more preferably no more than 5 weight percent and still more preferably no more than 1 weight percent, of structural units other than trimethylene carbonate. In embodiments in which the hydrophobic block is a homopolymer, the hydrophobic block is a poly(trimethylene carbonate) homopolymer (PTMC), which is formed upon heating or catalytic ring-opening of trimethylene carbonate. The structures of trimethylene carbonate, which is also known as 1,3-propylene carbonate, and the repeat unit of the PTMC homopolymer are shown below.

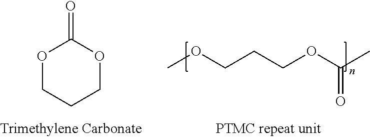

The hydrophilic block of the amphiphilic block copolymer comprises structural units of one or more alkylene glycols. Preferably, the hydrophilic block comprises, based on the total weight of the structural units used to form said hydrophilic block, no more than 10 weight percent, more preferably no more than 5 weight percent and still more preferably no more than 1 weight percent, of structural units other than said alkylene glycol(s). Preferably, the alkylene glycol structural units are selected from ethylene glycol, propylene glycol and mixtures thereof. In embodiments in which the hydrophilic block is a homopolymer, the hydrophilic block is a polyethylene glycol or a polypropylene glycol. More preferably, the hydrophilic polymer block comprises structural units of ethylene glycol, and most preferably is a polyethylene glycol homopolymer. As used herein structural units of ethylene glycol refer to —[OCH₂—CH₂]— repeat groups. Similarly, structural unit of propylene glycol refer to a —[OCH₂CH₂CH₂]— and/or —[OCH(CH₃)CH₂]— repeat groups.

Preferably, the amphiphilic block copolymer comprises a hydrophilic polymer block having a weight average molecular weight (M_{w}) ranging from about 1.25 kDa to about 3 kDa, more preferably from about 1.5 kDa to about 2.5 kDa.

Preferably, the amphiphilic block copolymer comprises a hydrophobic polymer block having a Mᵥ ranging from about 2.5 KDa to about 15 kDa, more preferably from about 4 kDa to about 10 kDa.

In particularly preferred embodiments, the amphiphilic block copolymer comprises from about 15% to about 60% of said hydrophilic polymer block, based on the M_{w} of the amphiphilic block copolymer.

According to a second aspect, the invention provides a drug delivery vector comprising one or more active pharmaceutical ingredients encapsulated within a nanoparticle according to the first aspect of the invention.

Reference herein to a drug delivery vector is to one or more active pharmaceutical ingredients encapsulated within a nanoparticle as defined above. Like the nanoparticle itself, the drug delivery vectors preferably have an average particle size, measured according to dynamic light scattering (DLS), from about 50 nm to about 500 nm, more preferably from about 75 nm to about 250 nm.

Reference herein to an active pharmaceutical ingredient or API (which can be used interchangeably with terms such as active agent, active ingredient, active pharmaceutical agent, and drug) refers any compound, composition of matter, or mixture thereof that provides a therapeutic or prophylactic effect when administered to a human or animal.

Examples of such APIs include, but are not limited to, antibiotics, analgesics, vaccines, anticonvulsants; antidiabetic agents, antifungal agents, anti-cancer agents, antiparkinsonian agents, antirheumatic agents, appetite suppressants, biological response modifiers, cardiovascular agents, central nervous system stimulants, contraceptive agents, dietary supplements, vitamins, minerals, lipids, saccharides, metals, amino acids (and precursors), nucleic acids and precursors, contrast agents, diagnostic agents, dopamine receptor agonists, erectile dysfunction agents, fertility agents, gastrointestinal agents, hormones, immunomodulators, antihypercalcemia agents, mast cell stabilizers, muscle relaxants, nutritional agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, anti-hyperthyroids, anti-asthmatics and vertigo agents. Preferably, said one or more API is an anti-cancer agent, and more preferably is selected from: alkylating agents such as cisplatin; mitotic inhibitors such as a gefitinib; and/or epigenetic modifiers such as a HDACi (histone deacetylase inhibitor) e.g. HDACi2 or DNA methyl transferase inhibitor (DNMTi). It has been surprisingly found that encapsulation of cisplatin in the nanoparticles of the invention results in improved cytotoxicity in cisplatin resistant cell lines OVCAR-3 wt and SKOV-3cis, in the latter of which WT IC₅₀ was restored by such encapsulation. Therefore, in particularly preferred embodiments said one or more API is an alkylating agent, and is most preferably cisplatin.

Additionally or alternatively, said one or more API is preferably selected from small (≤1000 daltons) molecule drugs, biopharmaceuticals (e.g. polysaccharides, polypeptides, proteins such as antibody, or nucleic acids) or any combination thereof. It has been found that drug delivery vectors of the present invention can readily accept large API payloads such as proteins in excess of 75 kDa. Therefore, where said one or more API is or includes a biopharmaceutical, said biopharmaceutical preferably is from about 3 kDa to about 75 kDa, and more preferably from about 5 kDa to about 65 kDa in size.

In particularly preferred embodiments said one or more API is selected from small molecule drugs and nucleic acids (e.g. miRNA). Further, as the skilled person would readily appreciate, such APIs may be provided in neutral form, or as pharmaceutically acceptable salt, solvate, ester, or prodrug thereof. As used herein, a pharmaceutically acceptable salt is understood to mean a compound formed by the interaction of an acid and a base, the hydrogen atoms of the acid being replaced by the positive ion of the base.

The nanoparticles of the present invention represent a flexible polymer system that is suitable for incorporating and improving the pharmacokinetic and phamacodynamic (PK/PD) profile of a wide range of APIs, and so represents an improved API delivery system. Such an improvement is particularly evident upon encapsulation of poorly soluble hydrophobic APIs. Therefore, in preferred embodiments, said one or more API includes one low solubility compound or agent (i.e. a compound or agent that requires 100 ml or more of water to dissolve 1 g of the drug or agent).

The polymer system is also suitable for encapsulating combinations of APIs, such as a combination of anticancer agents, preferably wherein each API has a different therapeutic mode of action. In a preferred example of this combined approach to API incorporation, the drug delivery vector comprises at least two, and optionally at least three, different co-encapsulated APIs, wherein each API has a different therapeutic mode of action. More preferably the drug delivery vector comprises at least two, and optionally all three, co-encapsulated anti-cancer agents selected from an epigenetic modifier, an alkylating agent, and a mitotic inhibitor. In particularly preferred examples, the drug delivery vector comprises an epigenetic modifier that is co-encapsulated with and one or both of an alkylating agent and a mitotic inhibitor. In exemplary embodiments, the drug delivery vector comprises a HDACi co-encapsulated with cisplatin and/or gefitinib. The inventors have surprisingly found that nanoformulations comprising a co-encapsulated combination of HDACi and cisplatin and/or gefitinib results in a synergistic enhancement of anticancer efficacy, particularly in the context of treating ovarian cancer.

Preferably, the weight ratio of said one or more active pharmaceutical ingredients: amphiphilic block copolymer is less than 1:3, and is preferably less than or equal to 1:4. The Applicants have surprisingly found that stable, nanoscopic vectors can be prepared comprising up to 20% or less than 25% active pharmaceutical ingredient, based on the weight of the amphiphilic block copolymer.

According to a third aspect of the invention there is provided a process for preparing a nanoparticle of the first aspect, the process comprising:
i. dissolving said amphiphilic block copolymer in an oligomeric alkylene glycol solution to prepare an alkylene glycol-polymer solution; and
ii. hydrating said alkylene glycol-polymer solution by addition of water, an aqueous solution or an aqueous buffer to prepare said nanoparticle.

In addition, according to a fourth aspect of the invention there is provided a process for preparing a drug delivery vector according to the second aspect by encapsulating one or more active pharmaceutical ingredients within a nanoparticle, wherein said process comprises:
i. co-dissolving said amphiphilic block copolymer and said one or more active pharmaceutical ingredients in an oligomeric alkylene glycol solution to prepare an alkylene glycol-polymer solution; and
ii. hydrating said alkylene glycol-polymer solution by the addition of water, an aqueous solution or an aqueous buffer to prepare said drug delivery vector.

Reference herein to an oligomeric alkylene glycol solution is to an oligomer comprising from 2 to 10, preferably from 2 to 8, and more preferably from 4 to 6 repeat units derived from ethylene glycol, propylene glycol or mixtures thereof. In preferred embodiments, the oligomeric alkylene glycol is an oligomeric ethylene glycol according to Formula (I), wherein n is an integer between 2 and 10, preferably an integer between 4 and 6.

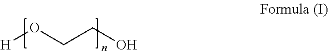

Preferably, the active pharmaceutical ingredient is an anti-cancer therapeutic, and is most preferably an alkylating agent such as cisplatin. Additionally or alternatively, the weight ratio of said active pharmaceutical ingredient: amphiphilic block copolymer is less than 1:3, and is preferably less than or equal to 1:4.

According to a fifth aspect of the invention, there is provided a pharmaceutical or veterinary composition comprising the drug delivery vector according to the fourth aspect of the invention and a pharmaceutically or veterinary acceptable diluent, carrier and/or excipient.

Suitable pharmaceutical or veterinary excipients are well known to those of skill in the art. Pharmaceutical or veterinary compositions may be formulated for administration by any suitable route, for example oral, buccal, intravenous intramuscular, nasal or bronchial (inhaled), subcutaneous, transdermal, or parenteral, and may be prepared by any methods well known in the art of pharmacy.

In preferred embodiments the composition is formulated for intravenous or intraperitoneal administration. In particularly preferred embodiments, the composition is formulated for intravenous administration.

The composition may be prepared by bringing into association the above defined vector with the carrier. In general, the formulations are prepared by uniformly and intimately bringing into association the vector with liquid carriers or finely divided solid carriers or both, and then if necessary shaping the product. The invention extends to methods for preparing a pharmaceutical composition comprising bringing a compound in the vector defined above in conjunction or association with a pharmaceutically or veterinary acceptable carrier or vehicle.

Formulations for oral administration in the present invention may be presented as: discrete units such as capsules, sachets or tablets each containing a predetermined amount of vector comprising the active agent; as a powder or granules; as a solution or a suspension of the vector comprising the active agent in an aqueous liquid or a non-aqueous liquid; or as an oil-in-water liquid emulsion or a water in oil liquid emulsion; or as a bolus etc.

For compositions for oral administration (e.g. tablets and capsules), the term “acceptable carrier” includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropylmethylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate, stearic acid, silicone fluid, talc waxes, oils and colloidal silica. Flavouring agents such as peppermint, oil of wintergreen, cherry flavouring and the like can also be used. It may be desirable to add a colouring agent to make the dosage form readily identifiable. Tablets may also be coated by methods well known in the art.

A tablet may be made by compression or moulding, optionally with one or more accessory ingredients. Compressed tablets may be prepared by compressing in a suitable machine the vector comprising the active agent in a free-flowing form such as a powder or granules, optionally mixed with a binder, lubricant, inert diluent, preservative, surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active agent.

Other formulations suitable for oral administration include lozenges comprising the active agent in a flavoured base, usually sucrose and acacia or tragacanth; pastilles comprising the active agent in an inert base such as gelatin and glycerin, or sucrose and acacia; and mouthwashes comprising the active agent in a suitable liquid carrier.

For topical application to the skin, the vector comprising the active agent may be made up into a cream, ointment, jelly, solution or suspension etc. Cream or ointment formulations that may be used for the drug are conventional formulations well known in the art, for example, as described in standard text books of pharmaceutics such as the British Pharmacopoeia.

Parenteral formulations will generally be sterile.

According to a sixth aspect, the invention relates to the drug delivery vector or the pharmaceutical or veterinary composition according to second or fifth aspect of the invention for use as a medicament.

According to a seventh aspect of the invention there is provided a drug delivery vector or pharmaceutical or veterinary composition as defined herein for use in the treatment of cancer.

According to an eighth aspect of the invention there is provided a drug delivery vector or pharmaceutical or veterinary composition as defined herein for use in the manufacture of a medicament to treat cancer.

In preferred embodiments the cancer referred to herein is a solid tumour cancer. As is readily appreciated by the skilled person, the term ‘solid tumour cancer’ includes but is not limited to, the following cancers: nasopharyngeal cancer, synovial cancer, hepatocellular cancer, renal cancer, cancer of connective tissues, melanoma, lung cancer, bowel cancer, colon cancer, rectal cancer, colorectal cancer, brain cancer, throat cancer, oral cancer, liver cancer, bone cancer, pancreatic cancer, choriocarcinoma, gastrinoma, pheochromocytoma, prolactinoma, neuroma, von Hippel-Lindau disease, adrenal cancer, anal cancer, bile duct cancer, bladder cancer, ureter cancer, brain cancer, oligodendroglioma, neuroblastoma, meningioma, spinal cord tumour, osteochondroma, chondrosarcoma, Ewing's sarcoma, cancer of unknown primary site, carcinoid, carcinoid of gastrointestinal tract, fibrosarcoma, breast cancer, Paget's disease, cervical cancer, oesophagus cancer, gall bladder cancer, head cancer, eye cancer, neck cancer, kidney cancer, Wilms' tumour, Kaposi's sarcoma, prostate cancer, testicular cancer, oral cancer, skin cancer, mesothelioma, ovarian cancer, glucagonoma, parathyroid cancer, penis cancer, pituitary cancer, soft tissue sarcoma, retinoblastoma, small intestine cancer, stomach cancer, thymus cancer, thyroid cancer, trophoblastic cancer, uterine cancer, endometrial cancer, vagina cancer, vulva cancer, acoustic neuroma, insulinoma, carcinoid syndrome, somatostatinoma, gum cancer, heart cancer, lip cancer, meninges cancer, mouth cancer, nerve cancer, palate cancer, parotid gland cancer, peritoneum cancer, pharynx cancer, pleural cancer, salivary gland cancer, tongue cancer and tonsil cancer.

More preferably, the solid tumour cancer is selected from: testicular cancer, ovarian cancer, cervical cancer, breast cancer, bladder cancer, head and neck cancer, esophageal cancer, lung cancer, mesothelioma, brain tumors and neuroblastoma. In exemplary embodiments, the solid tumour cancer is ovarian cancer.

The nanoparticles of the invention provide a drug delivery platform that is compatible with IP delivery. In particular, biodistribution and accumulation studies have been carried out following IP administration of cisplatin encapsulated nanoparticles of the invention into balb/c mice with SKOV-3luc subcutaneous tumour xenograft lumpectomies, and have found, in contrast to many IP administered drugs that show clearance in under 24 hours, a strong nanoparticle signal was observed in the abdominal cavity (peritoneum) throughout the (48 hour) duration of these studies, in addition to a strong signal in the flank tumour lumpectomy after 4 hours. Therefore, in certain preferred embodiments the nanoparticles of the present invention provide a drug delivery platform for chemotherapeutic drugs that are administered via intraperitoneal injection, and more preferably by pressurized Intraperitoneal Aerosolized Chemotherapy PIAC), to treat solid tumour cancers. Cancers that are treated by PIAC include, but are not limited to, ovarian, uterine, gastric, colorectal and appendiceal cancers. Further, PIAC may be utilized to treat pancreatic or liver cancers that have spread to the abdomen.

According to a ninth aspect, the invention relates to a method of treatment comprising administering to a subject in need thereof an effective amount of a drug delivery vector or a pharmaceutical or veterinary composition according to the second or fifth aspect of the invention.

Preferably said subject has cancer and the drug delivery vector comprises an anti-cancer therapeutic. More preferably, the subject has a solid tumour cancer as described herein, and still more preferably the subject has ovarian cancer. In particularly preferred embodiments, the anti-cancer therapeutic is cisplatin.

Reference herein to an “effective amount” of the drug delivery vector or a pharmaceutical or veterinary composition comprising same is one that is sufficient to achieve a desired biological effect, such as cancer cell death. It is understood that the effective dosage will be dependent upon the age, sex, health, and weight of the recipient, kind of concurrent treatment, if any, frequency of treatment, and the nature of the effect desired. Typically, the effective amount is determined by those administering the treatment.

Throughout the description and claims of this specification, the words “comprise” and “contain” and variations of the words, for example “comprising” and “comprises”, mean “including but not limited to” and do not exclude other moieties, additives, components, integers or steps. However, in preferred embodiments, the words “comprise” and “contain” and variations of the words, mean “consisting only of” and, as a consequence, other moieties, additives, components, integers or steps are thereby excluded.

Throughout the description and claims of this specification, the singular encompasses the plural unless the context otherwise requires. In particular, where the indefinite article is used, the specification is to be understood as contemplating plurality as well as singularity, unless the context requires otherwise.

All references, including any patent or patent application, cited in this specification are hereby incorporated by reference. No admission is made that any reference constitutes prior art. Further, no admission is made that any of the prior art constitutes part of the common general knowledge in the art.

Preferred features of each aspect of the invention may be as described in connection with any of the other aspects.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith. Moreover, unless stated otherwise, any feature disclosed herein may be replaced by an alternative feature serving the same or a similar purpose.

The invention will now be described by way of example only with reference to the Examples below and to the following Figures wherein:

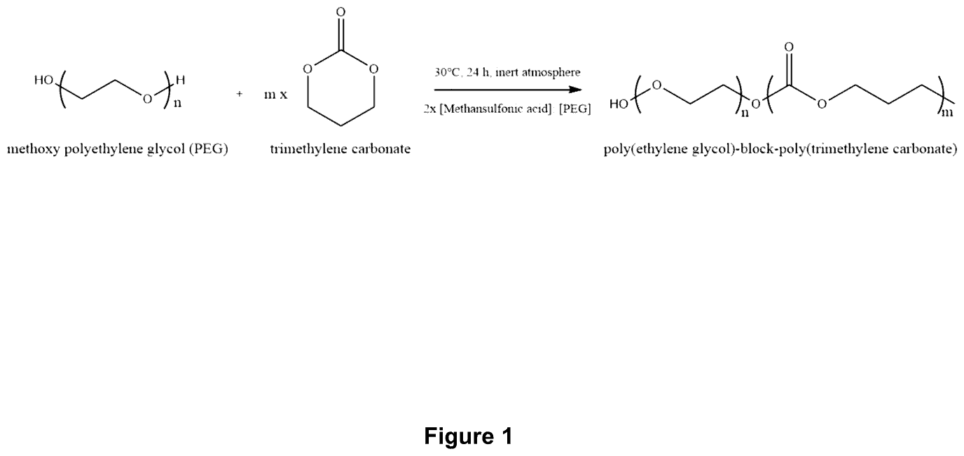

FIG. 1. PEG-PTMC synthesis schematic.

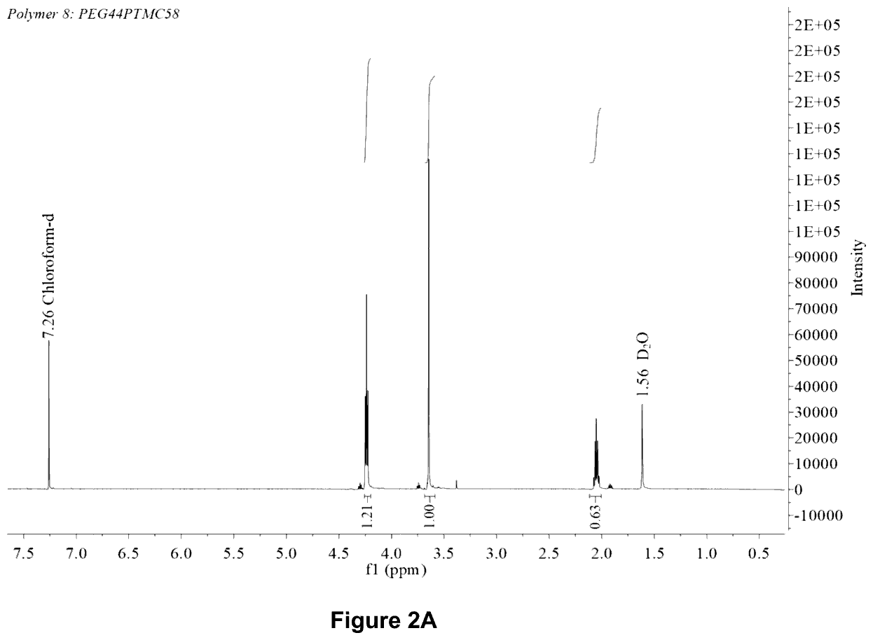

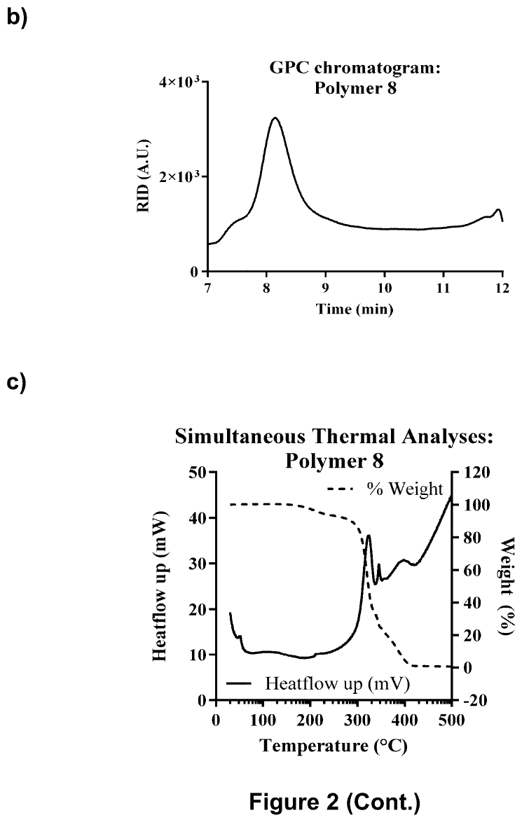

FIG. 2. Material characterization of polymer 8 as a bulk material. ¹H NMR of polymer 8 dissolved in CDC₃ using 500 MHz Bruker NMR (a), gel permeation chromatography (GPC) using 100% THF as mobile phase and a flow rate of 1 mL min-1 (b), chromatogram trimmed to region elution period. Simultaneous differential scanning calorimetry and thermal gravimetric analysis (c) was carried out on 5-10 mg of material.

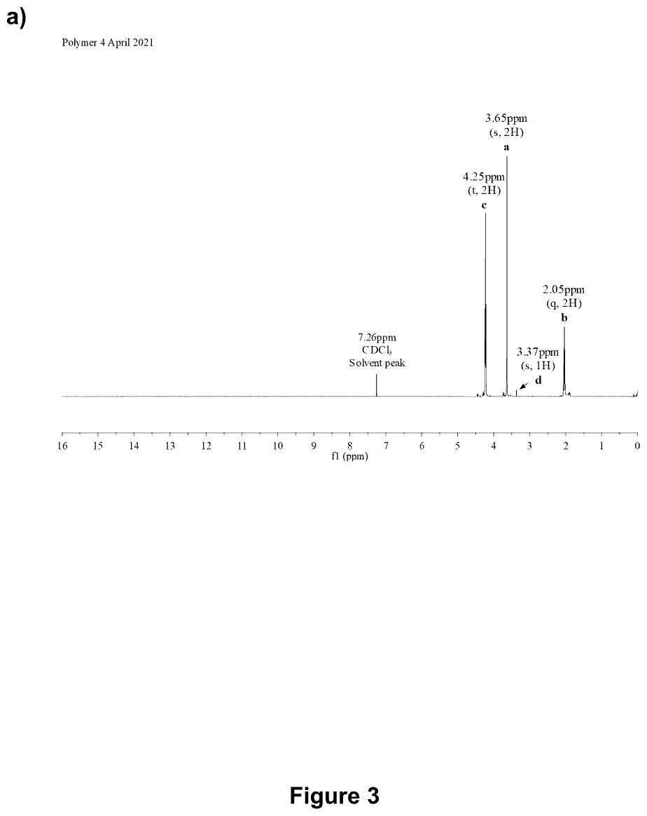

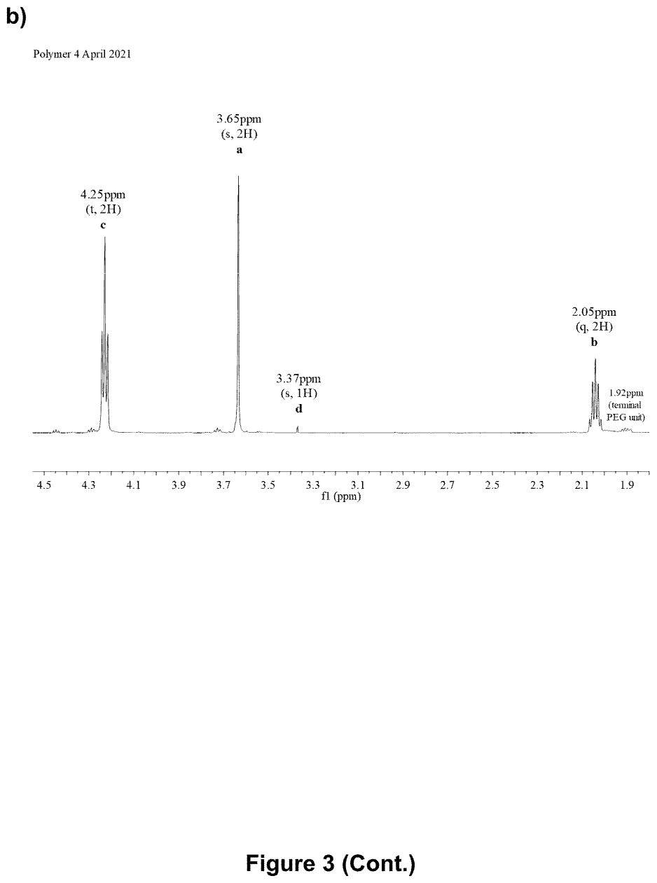

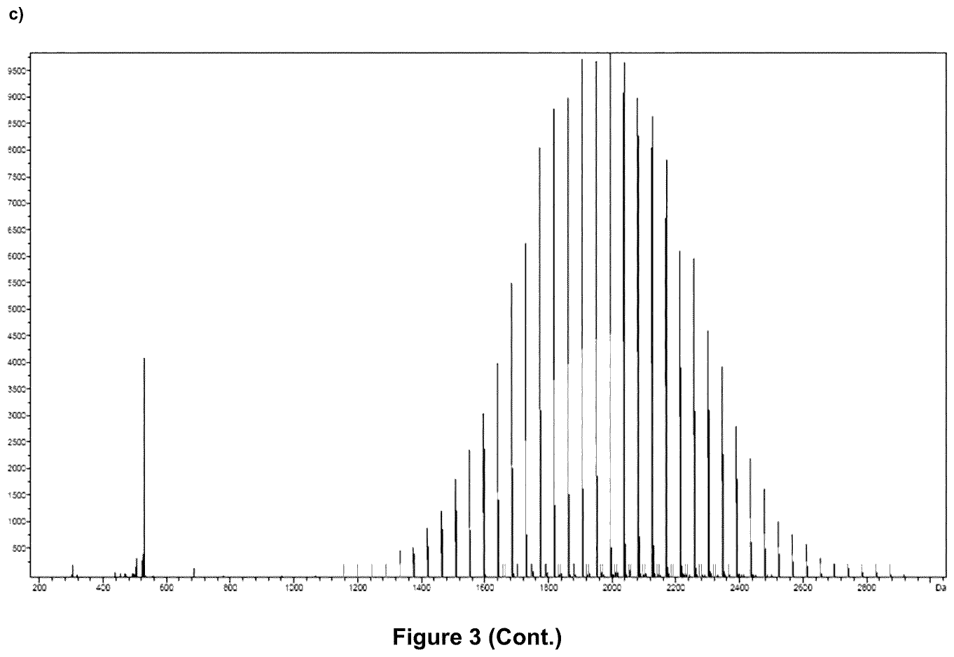

FIG. 3. ¹H NMR of PEG-PTMC polymer 4. Full NMR spectra showing deuterated solvent and water peaks (a), zoom of spectra between 1.8 and 4.5 ppm, showing detailed peaks including splitting and annotated molecule (b). Example of Matrix-assisted laser desorption/ionization-Time of Flight Mass Spectrometry (MALDI-TOF MS) results from PEG sample (c).

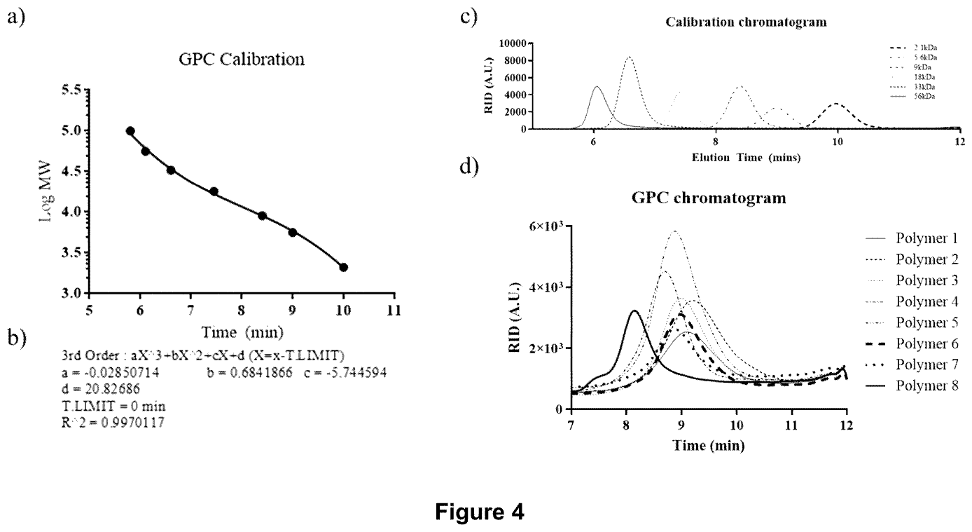

FIG. 4. GPC output for polymer characterization. Calibration curve, polynomial fit details including R\hat{ }2 values and chromatogram overlay of standards shown in panels a-c, respectively. Polymer chromatogram of region of interest given in panel d.

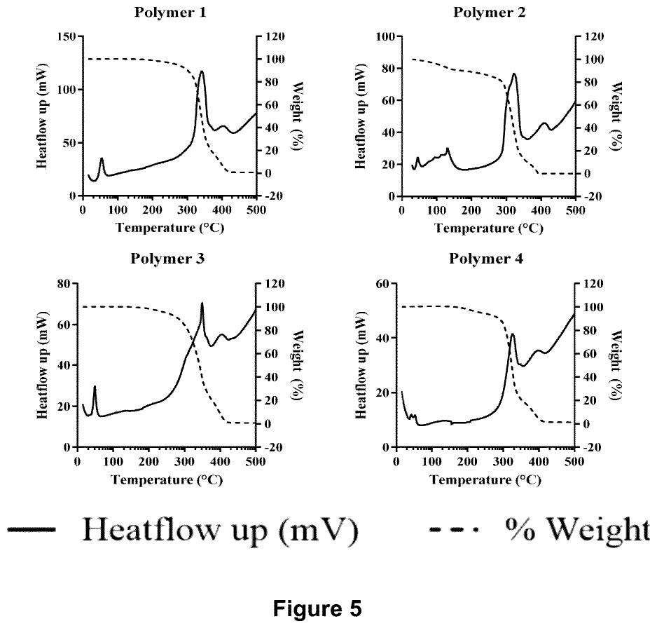

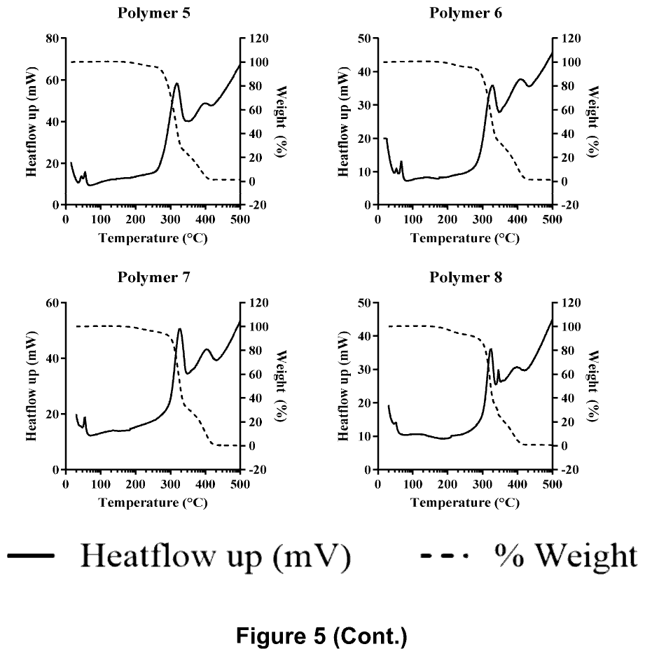

FIG. 5. Thermal analysis of polymers. Simultaneous differential scanning calorimetry and thermal gravimetric analysis was carried out on 5-10 mg of material.

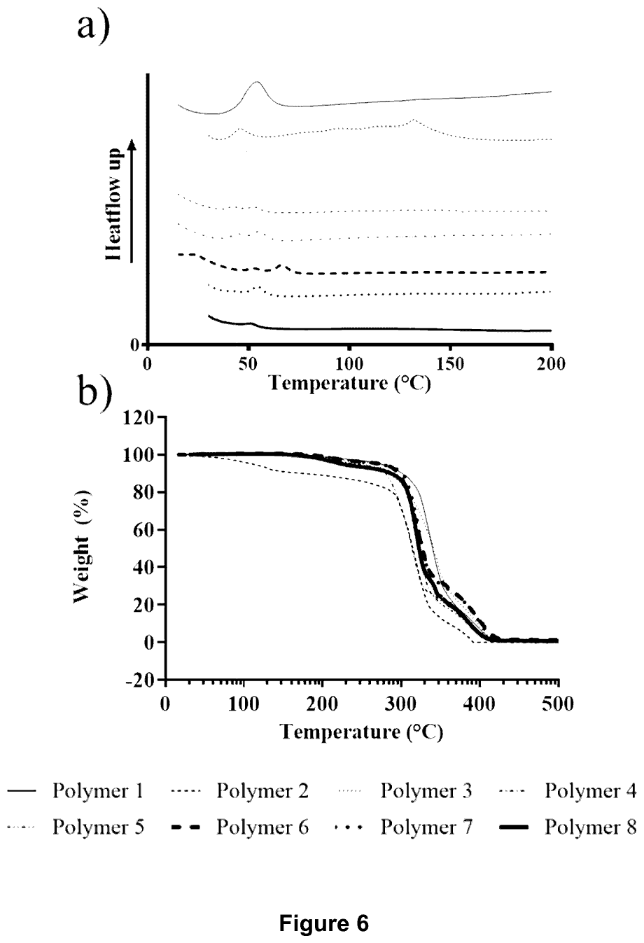

FIG. 6. Thermal analyses of polymers. Differential scanning calorimetry from RT to 200° C. (a). Thermal gravimetric analysis of polymers from RT to 500° C. (b).

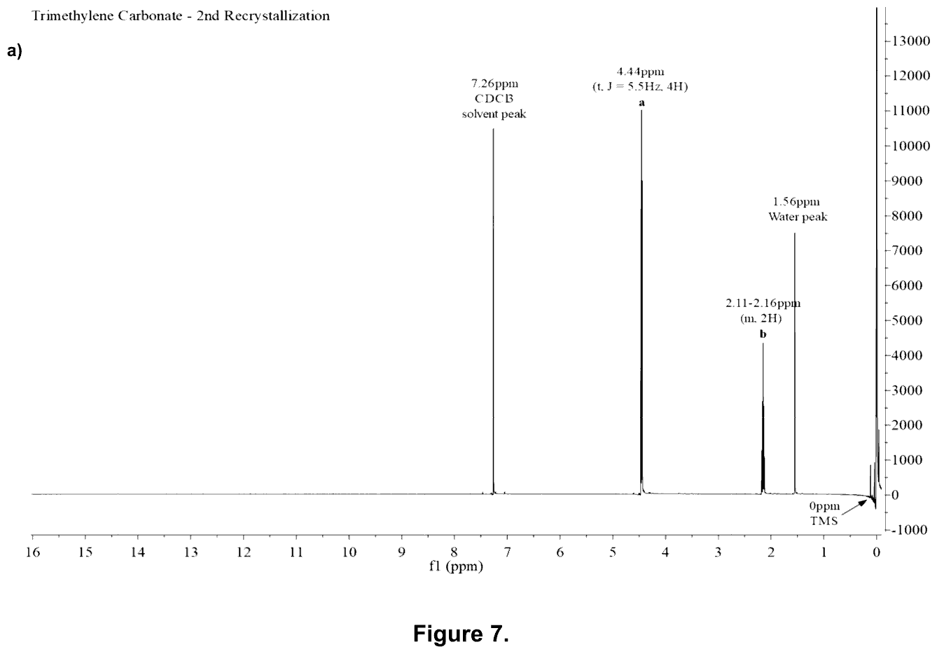

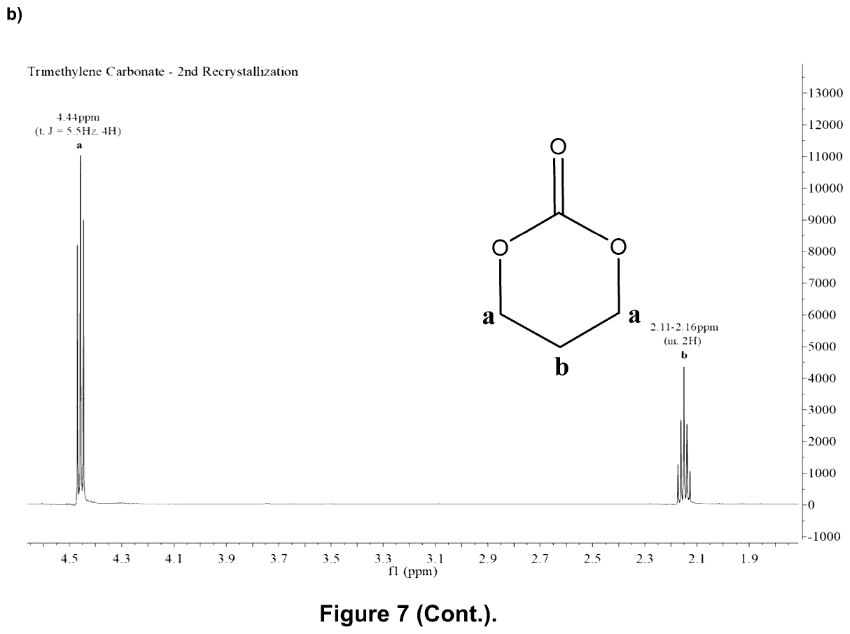

FIG. 7. ¹H NMR of trimethylene carbonate (monomer) run in CDC3 with 1% (v/v) Tetramethylsilane(TMS). Full NMR spectra showing deuterated solvent and water peaks (a), spectra between 1.8 and 4.6 ppm showing region of interest, showing detailed peaks including splitting and annotated molecule (b).

FIG. 8. Nanoparticle sizes characterized by DLS, averages of three repeats used for plot, all polymers (a), NP4(b), NP7(c) and NP8(d).

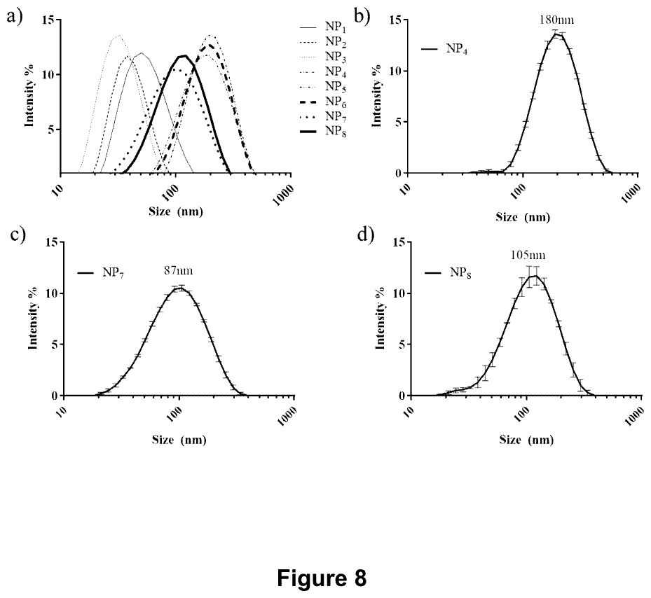

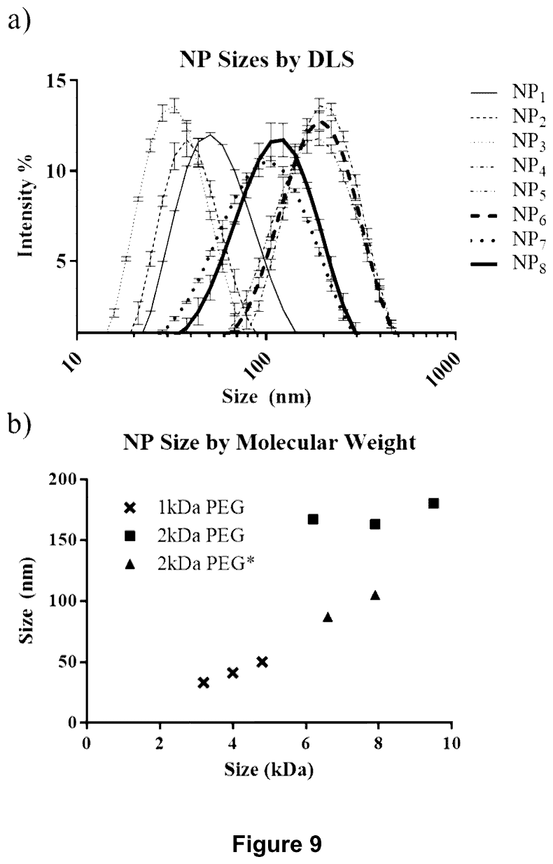

FIG. 9. Polymer molecular weight and effect on NP size. NP size as determined by DLS, including SEM error bars (a). Relationship between molecular weight of the polymer and the size of the NP formulation (b). Samples are identified by the PEG molecular weight, 2 kDa PEG* denotes PEG acquired from SIGMA in contrast to the other two which were acquired from PEGworks.

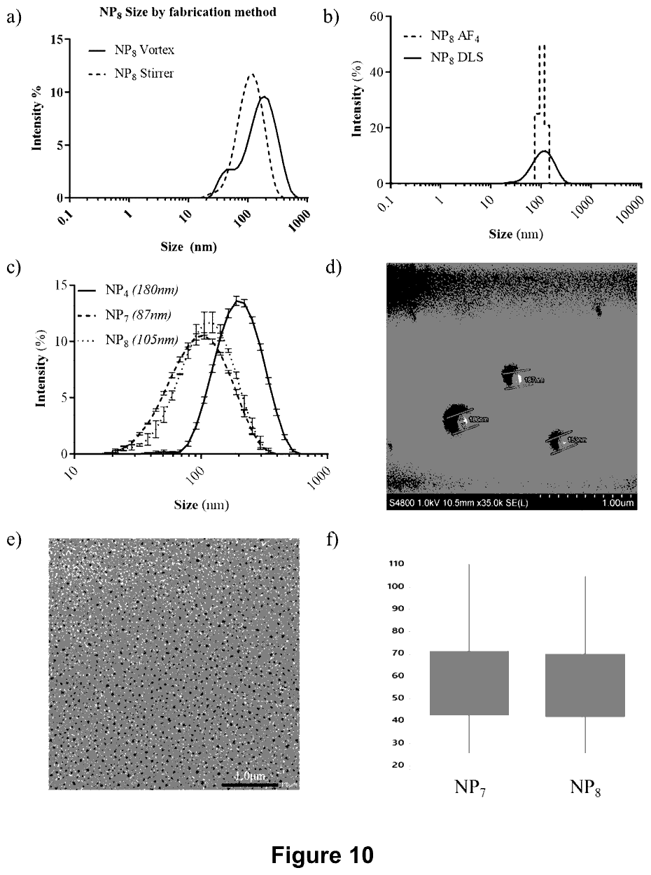

FIG. 10. Effect of fabrication method and characterization technique on NP size. NP₈ size by fabrication method (a), comparison of NP₈ size intensity as measured by DLS and AF₄ (b) NPs_{4,7,8} size by DLS (c) for comparison with SEM (d) and TEM (e; example image of NPs), size distribution based on TEM of NP₇ and NP₈ (f).

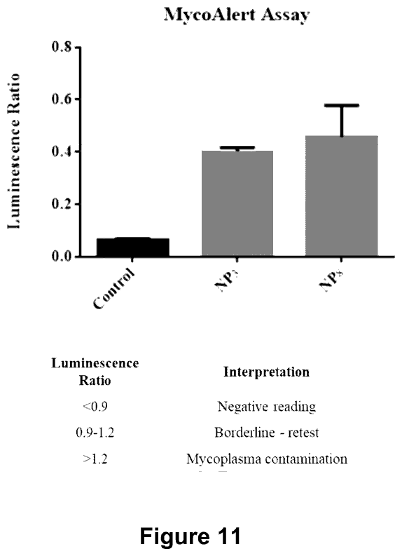

FIG. 11. Results of MycoAlert assay, samples were tested in duplicate.

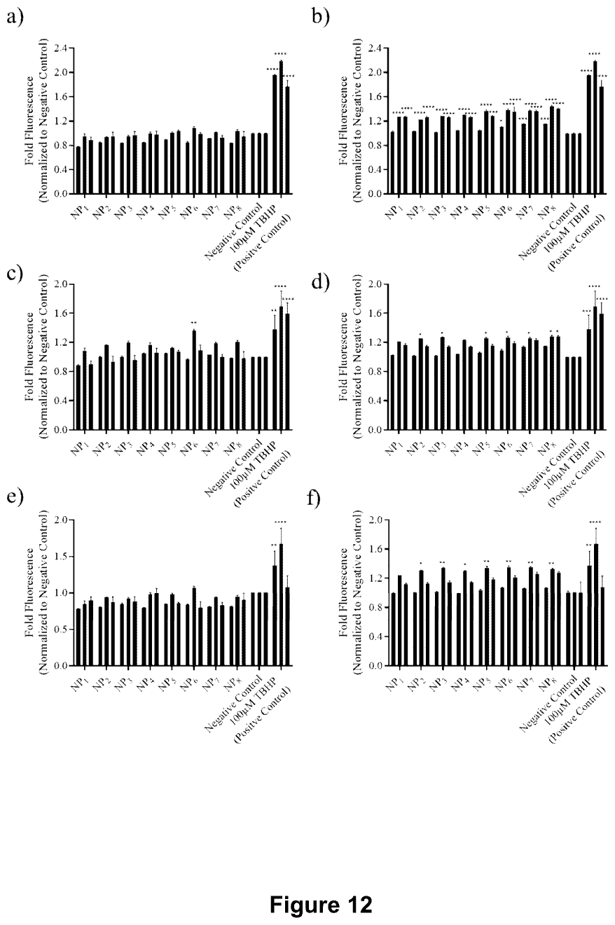

FIG. 12. ROS analysis in solid tumour cancers. ROS analysis in OVCAR-3 treated with “clinically relevant” dose (a) and “forced” dose (b); SKOV-3 treated with “clinically relevant” dose (c) and “forced” dose (d); as determined for the in vivo study. ROS analysis in control cell line, HeLa treated with “clinically relevant” dose (e) and “forced” dose (f). Data is grouped by treatment, (n=3). Each column represents the mean value for each timepoint investigated, time points were 4, 24 and 48 h. Two-way ANOVAs with Dunnett's multiple comparison test for statistical significance was performed (*p<0.05, **p<0.01, ***p<0.001, ****p<0.0001) comparing all samples to negative controls.

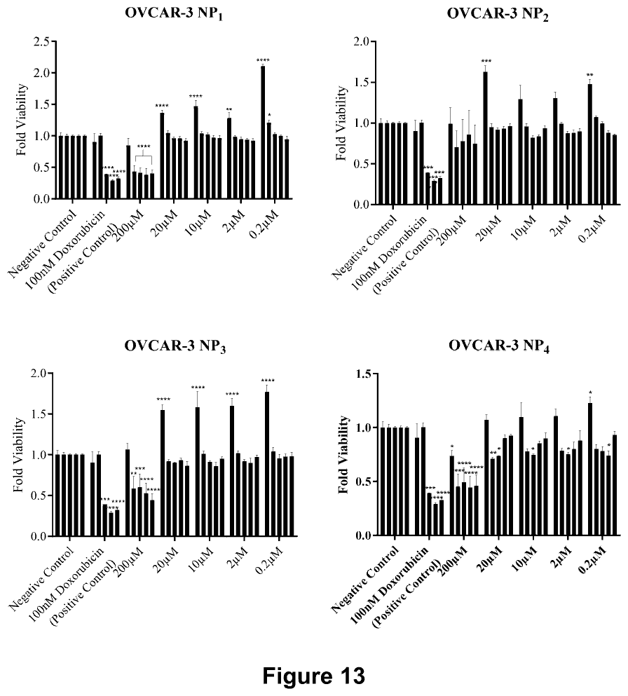

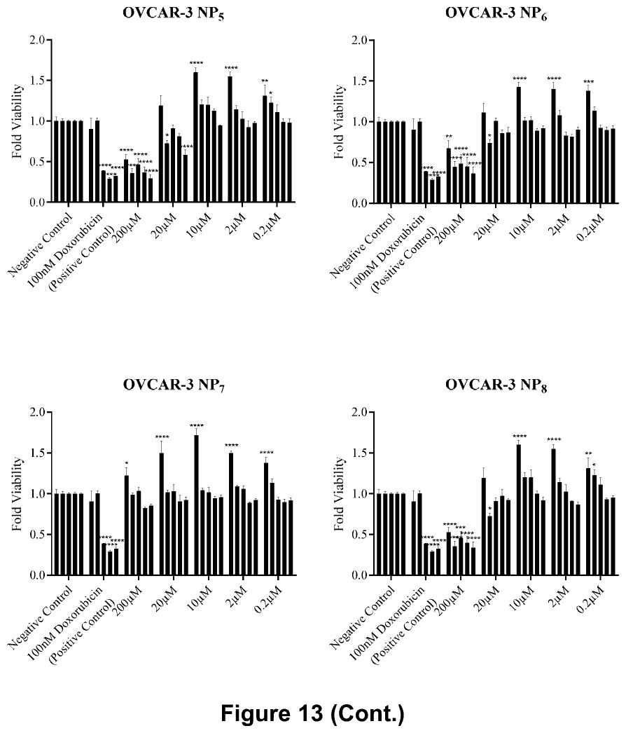

FIG. 13. RT-Glo viability results for Empty NPs 1-8(a-h, respectively) at 0.2, 2, 10, 20 and 200 μM in OVCAR-3 over 96 h (n=3). All data has been normalized to the untreated control cells. Bars are clustered showing 0, 24, 48, 72 and 96 h, respectfully and error bars are SEM.

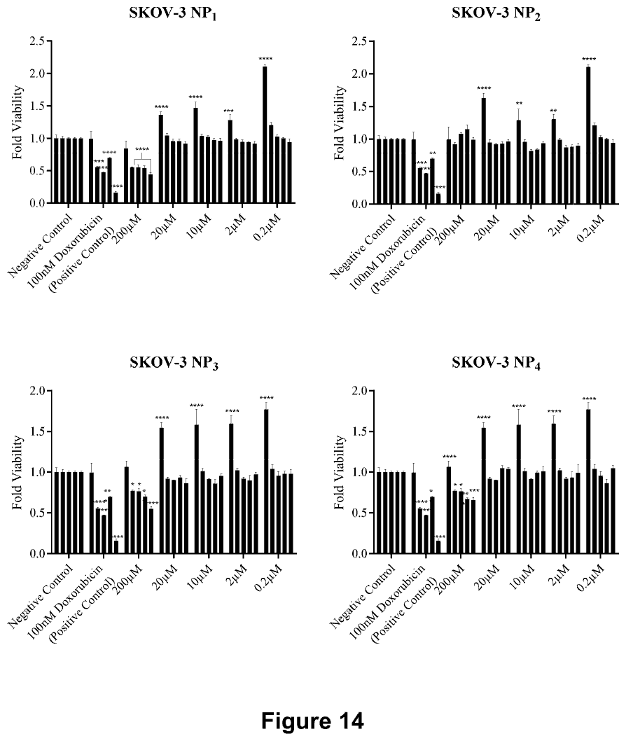

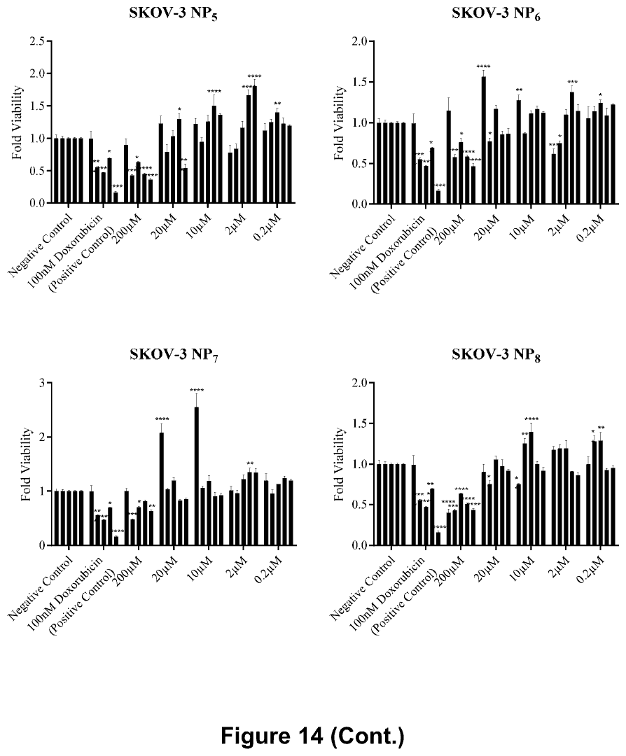

FIG. 14. RT-Glo viability results for Empty NPs 1-8 (a-h, respectively) at 0.2, 2, 10, 20 and 200 μM in SKOV-3 over 96 h (n=3). All data has been normalized to the untreated control cells. Bars are clustered showing 0, 24, 48, 72 and 96 h, respectfully and error bars are SEM. Two-way ANOVAs with Dunnett's multiple comparison test for statistical significance was performed (*p<0.05, **p<0.01, ***p<0.001, ****p<0.0001) comparing all samples to negative controls. Where samples had a significant increase in viability, grey symbols were used in contrast to black.

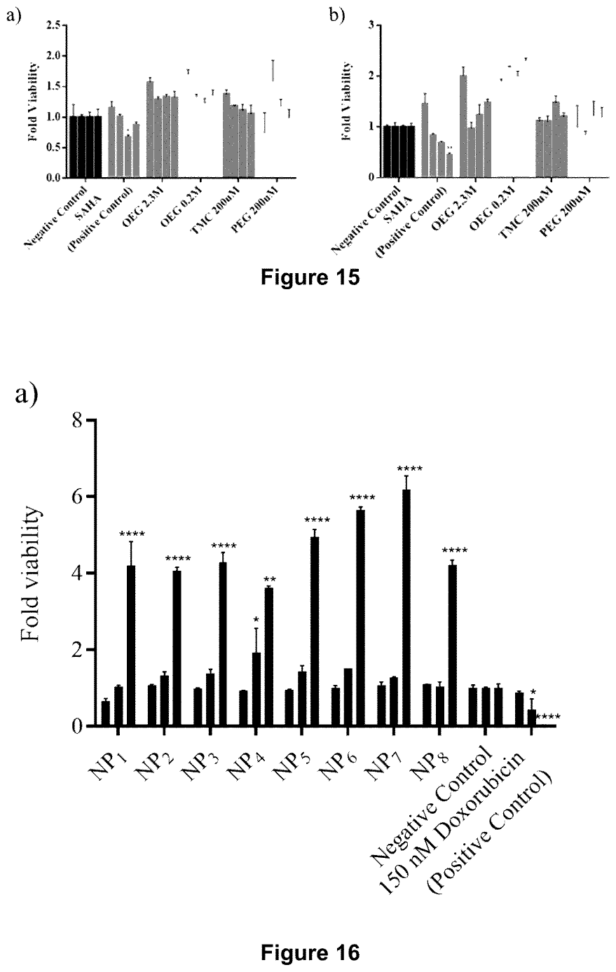

FIG. 15. RT-Glo viability results for NP components in OVCAR-3 (a) and SKOV-3 (b) over 72 h (n=3). All data has been normalized to the untreated control cells. Bars are clustered showing 0, 24, 48 and 72 h, respectfully and error bars are SEM. Two-way ANOVAs with Dunnett's multiple comparison test for statistical significance was performed (*p<0.05, **p<0.01, ***p<0.001, ****p<0.0001) comparing all samples to negative controls. Where samples had a significant increase in viability, grey symbols were used in contrast to black.

FIG. 16 3D cell viability results for Empty NPs 1-8 at 25 μM in HeLa(n=2). All data has been normalized to the untreated control spheroids. Bars are clustered showing 24, 48 and 72 h, respectfully and error bars are SEM. Two-way ANOVAs with Dunnett's multiple comparison test for statistical significance was performed (*p<0.05, **p<0.01, ***p<0.001, ****p<0.0001) comparing all samples to negative controls. Where samples had a significant increase in viability, grey symbols were used in contrast to black.

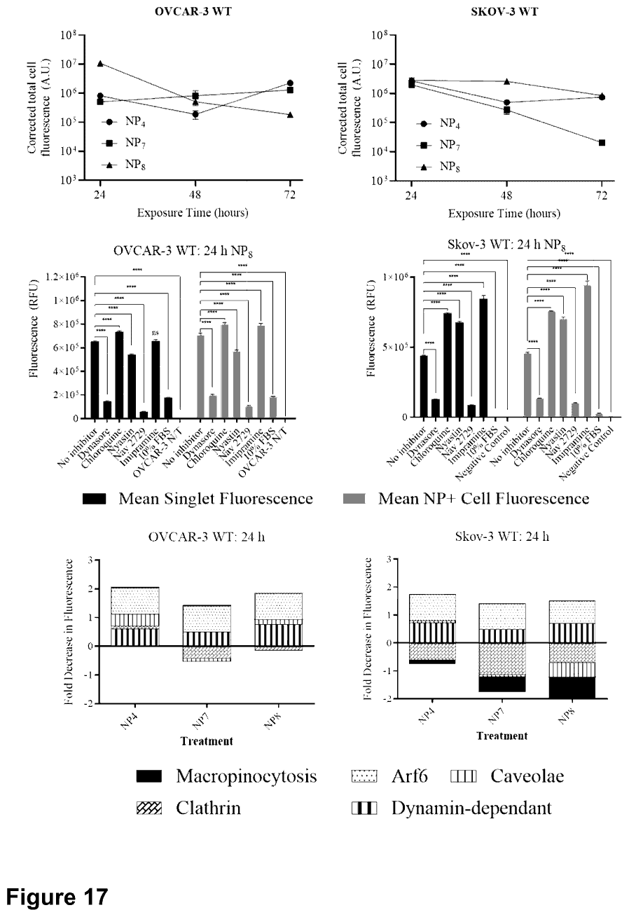

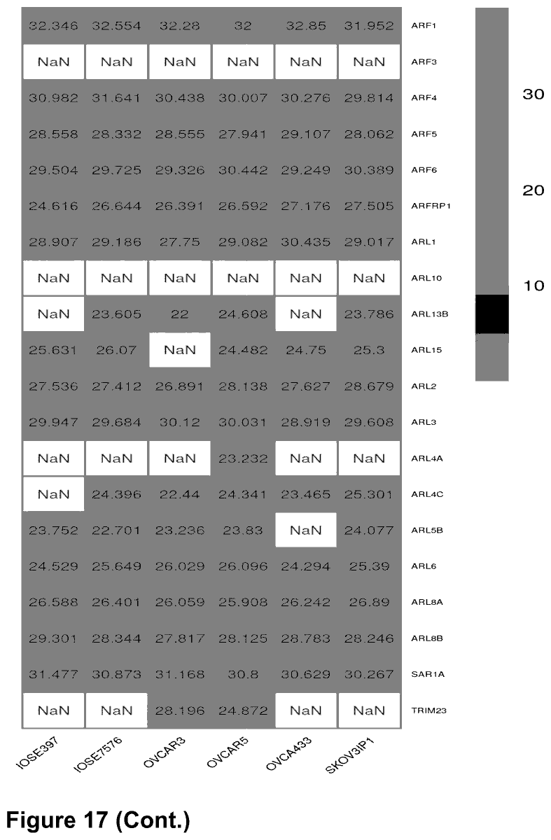

FIG. 17. NP uptake over time in OVCAR-3 wt (a) and SKOV-3 wt (b), endocytosis results, raw fluorescence change of 24 h NP₈ treatments in OVCAR-3 WT (d) and SKOV-3 WT (e) with matching stacked column graphs highlighting proportional responses (e) and (f). Heatmap of arf6 protein in select OC cell lines, showing a ubiquitously high expression across healthy and HGSOC (g).

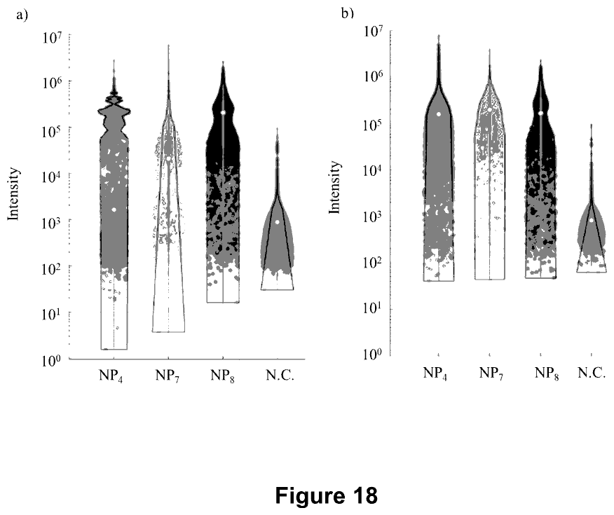

FIG. 18. NP uptake in OVCAR-3 (a) and SKOV-3 (b) spheroids. Intensity measured on the B6 channel(excitation 624 nm; emission 687 nm). Mean value is denoted by the white marker, grey lines illustrate the interquartile range.

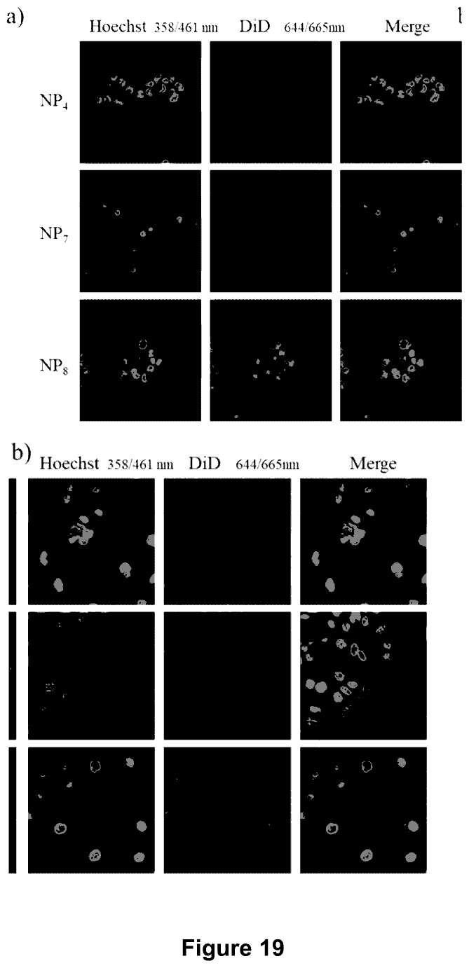

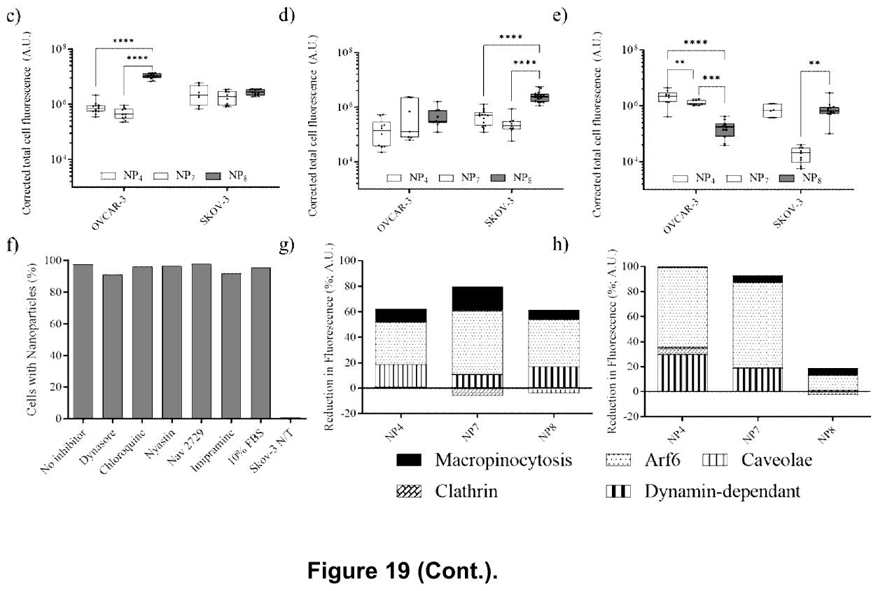

FIG. 19 NP(DiD) uptake following 24 h exposure in OVCAR-3 (a) and SKOV-3 (b). Uptake quantification measured using corrected total cell fluorescence method and plotted for 24 (c), 48 (d) and 72 h (e) NP(DiD) treatments. Endocytosis elucidation conducted using chemical inhibitors, prior to NP(DiD) treatments and cells analyzed using flow cytometry. Example of cell population positive for NP₈ in SKOV-3 WT (f) and endocytosis pathway distribution graphs for OVCAR-3 WT (g) and SKOV-3 (h).

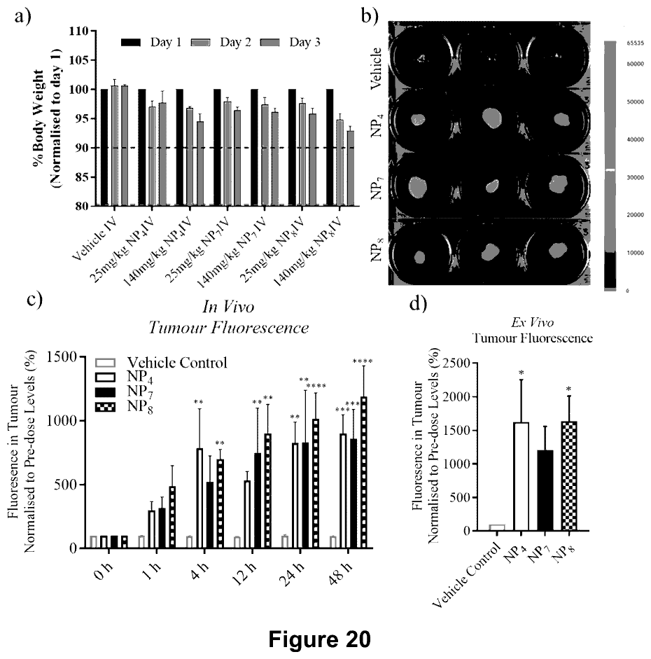

FIG. 20. Tolerance and tumour accumulation for PEG-PTMC Nanoparticles. Body weight summary (a) includes humane standards threshold for early termination. Ex vivotumour images (b) in vivoquantification of NP uptake(c) and quantified ex vivovalues (D) (n=3). Significance indicated by asterixis using standard procedure.

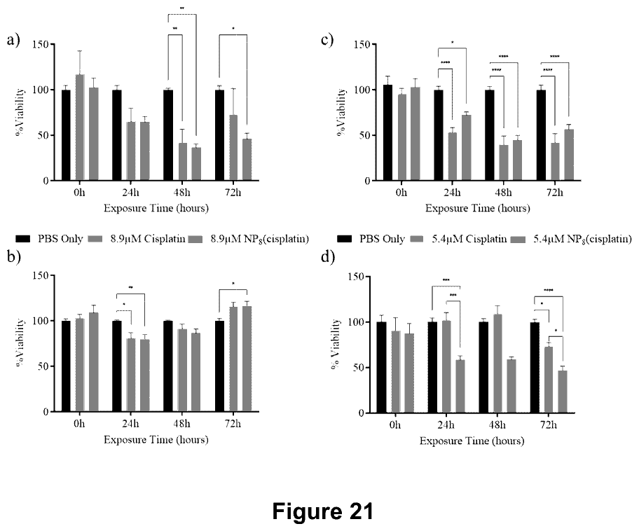

FIG. 21. Viability assessment of free drug cisplatin (CisPt) and NP(CisPt) in OC models in conventional monolayer growth conditions Results for OVCAR-3 wt (a), SKOV-3 wt (b), OVCAR-3cis (c) and SKOV-3cis (d) All data has been normalized to the untreated control in each repeat. Three biological repeats with five technical repeats per biological rep. Error bars are standard error of the means. Two-way ANOVAs with Dunnett's multiple comparison test for statistical significance was performed (*p<0.05, **p<0.01, ***p<0.001, ****p<0.0001) comparing all samples.

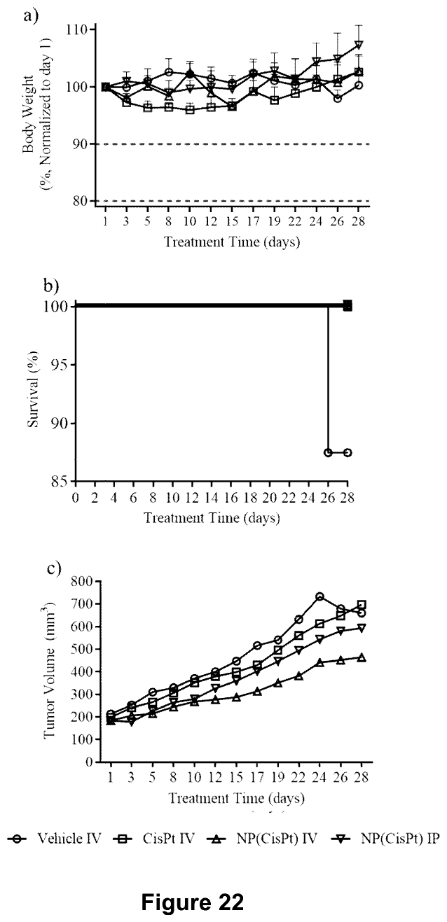

FIG. 22. 28-day single dose efficacy study in female balb/c with SKOV-3luc subcutaneous tumor xenograft (n=6). Cisplatin treatments (free drug and NP(CisPt)) were 5 mg/kg of platinum. Mean body weight measurements (a), survival curve (b) and mean tumour volume (c) graphs. Error bars included in (a) are SEM. Error bars excluded for (c) for ease of graph interpretation, these are included in supplementary materials.

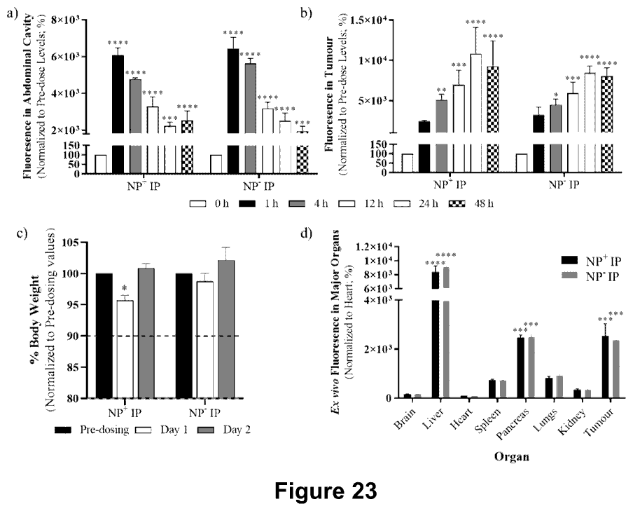

FIG. 23. Tolerance and tumor accumulation for 25 mg/kg single dose, IP administration of PEG-PTMC NPs (n=3). In vivo quantification of NP accumulation in abdominal cavity (peritoneum; a) and SKOV-3luciferase subcutaneous tumor xenograft (b). Body weight summary (c) including early termination threshold (red dashed line) based on humane standards. Quantified ex vivo fluorescence values from resected major organs (d). One-way ANOVA with Dunnett's multiple comparison test for statistical significance was performed (*p<0.05, **p<0.01, ***p<0.001, ****p<0.0001). comparing treatments to pre-dose values (a-b), day 1 values (c) or control tissue values (heart; d).

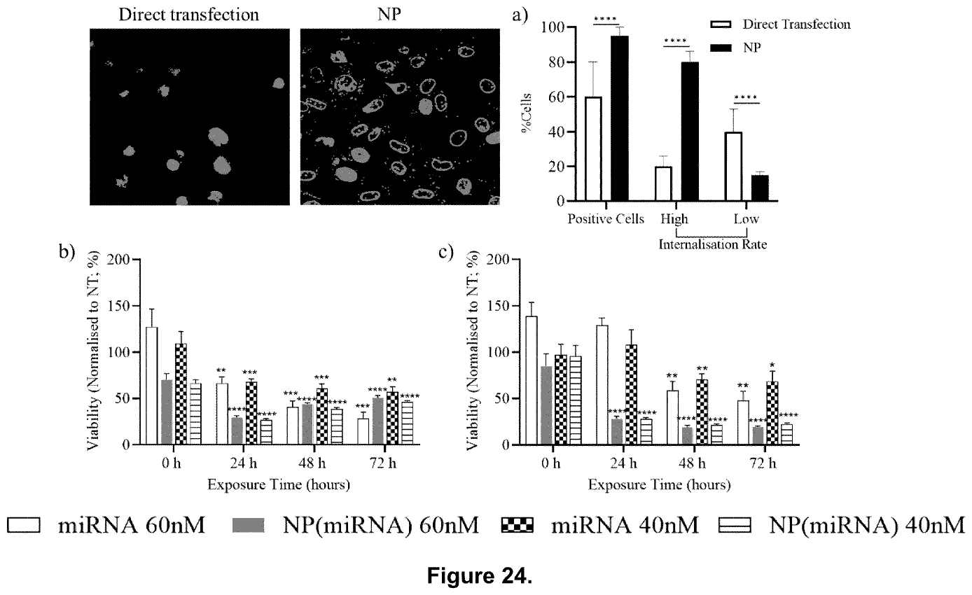

FIG. 24. Comparison of direct transfection and nano-encapsulated miRNA. Uptake study conducted in SKOV-3 wt cells using fluorescently conjugated miRNA administered by direct transfection or nanoparticle, Hoescht used as nuclear co-stain. Images quantified and plotted (a), defining total number of cells positive for fluorescence and binary binning of high or low signal. Anti-cancer efficacy of miR-28-5p following standard direct transfection and nano-enabled drug delivery in SKOV-3 wt (b) and OVCAR-3 wt (c). One-way ANOVA with Dunnett's multiple comparison test for statistical significance was performed (*p<0.05, **p<0.01, ***p<0.001, ****p<0.0001).

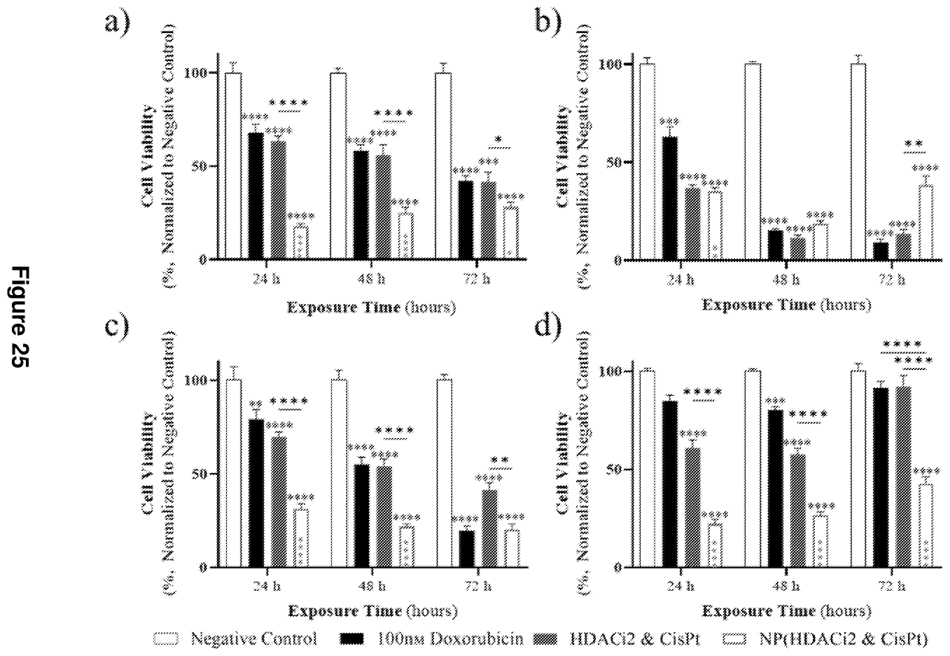

FIG. 25. Combination treatments of HDACi2 with cisplatin in SKOV-3 wt (a), SKOV-3cis (b), OVCAR-3 wt (c) and OVCAR-3cis (d). All data has been normalized to the untreated control in each repeat. Three biological repeats with three repeats per biological rep. Error bars are standard error of the means. Two-way ANOVAs with Dunnett's multiple comparison test for statistical significance was performed (*p<0.05, **p<0.01, ***p<0.001, ****p<0.0001). Asterix used to denote significance in the following scheme: against untreated control (when above bars), between experimental groups (lines and asterisks) and against positive control doxorubicin (red asterisks at base of bar).

Table 1. Treatment groups for 28-day in vitro efficacy study.

Table 2. Collated characterization data for polymers 1-8, including NP size, and zeta potential measurements. GPC tailing and D given to 1d.p.

Table 3. Polystyrene standards used in GPC calibration Table 4. Luminescence ratio interpretation ranges as provided by MycoAlert instructions.

Table 5. Microtissue penetration quantitation values for all cell lines investigated.

### Methods and Materials

### Instrumentation & Analysis.

A 500 MHz Bruker nuclear magnetic resonance (NMR) instrument was used to collect all 1H spectra and Nuclear Overhauser Effect Spectroscopy (NOESY) analyses. CDC₃ was used as the solvent for monomer and polymer analyses, HDO or deuterated PBS (0.01 M, pH 7.4) was used during NOESY analysis of nanoparticles. Output spectra analysed in MestraNova. Polydispersity index of synthesized polymers was measured on a Shimadzu Prominence gel permeation chromatography (GPC) system equipped with LC-20AT pump, SIL-20A HT autosampler, CTO-20A column oven running tandem Phenogel™ 5 μM guard and Phenogel™ 5 μM 10E3 Å columns to RID-20A refractory index detector and SPD-20AV dual wavelength detector (190-900 nm). HPLC grade tetrahydrofuran (THF) was used as the eluent at a flow rate of 1 mL/min and polystyrene standards (PSS Polymer, Germany) for calibration. Size, polydispersity and zeta potential of particles were measured using a Malvern Zetasizer Nano ZS dynamic light scattering (DLS) system with Zetasizer 8.01.4906 software (Malvern Panalytical) using non-invasive backscattering (NIBS) 173° configuration and He—Ne laser (633 nm). Comparative size analysis was conducted by asymmetric field-flow-field fractionation (AF4) over 10 kDa regenerated cellulose and 350 μM spacer coupled to multi-angle light scattering (MALS) unit. Wyatt Eclipse Dualtec instrument in line with a Shimazu LC-335 20A Prominence system with CTO20A injector connected to a Wyatt DAMN 336 HELEOS II light scattering detector (12.9°, 20.6°, 29.6°, 37.4°, 44.8°, 53.0°, 61.1°, 337 0.1°, 80.1°, 90.0°, 99.9°, 109.9°, 120.1°, 130.5°, 149.1°, and 157.8° preset angles).

Calibrations were run using bovine serum albumin and data analysed using Zimm model on Astra 6.1.1. Thermal analysis of polymers was conducted using a Perkin Elmer Simultaneous Thermogravimetric Analyzer (STA) 6000 connected to a recirculating water chiller functioning at 15° C. Samples were measured from ambient temperatures up to a maximum of 995° C. under a constant nitrogen flow of 20 mL/min. Size and morphological analysis of particles was conducted using AFM and Hitachi S4800 Field Emission Scanning Electron Microscope. Fluorescence and luminescence measurements were obtained using FLUOstar omega microplate reader. Confocal laser scanning microscopy (CLSM) images were obtained using Zeiss LSM710 fluorescent confocal microscope (Carl Zeiss Microscopy, Jena) and Zen system software (Black edition) using 543 nm and 405 nm laser lines. Flow cytometry was conducted using an Amnis® CellStream® benchtop flow cytometry system equipped with 375 nm, 350 nm and 488 nm lasers, a dedicated side scatter laser (785 nm) and dedicated forward scatter LED (450 nm) coupled to a filter stack for spatial separations of photons (456/51, 528/46, 583/24, 611/31, 702/87, and 773/56 nm).

### Monomer Synthesis:

Synthesis of trimethylene carbonate (TMC) was carried out as described in literature [15-17]. Ethyl chloroformate (15 mL, 176.4 mmol), 1,3-propandiol (6.4 mL, 88.2 mmol) and dry tetrahydrofuran (THF, 100 mL) were combined and put on ice. In a separate flask, triethylamine (24.6 mL, 176.4 mmol) was dissolved in dry THF (25 mL) and added dropwise to the initial mixture. The reaction was stirred at room temperature for 3 hours in an inert atmosphere. Triethylammonium chloride salts were removed by vacuum filtration. Filtrate was concentrated under reduced pressure before overnight recrystallization in warm THF and ice-cold diethyl ether (volume ratio 2:9). Solids were dried, dissolved in 1,4-dioxane and lyophilized. Composition was determined by 1H NMR (500 MHz, CDCl3, δ) 2.11-2.16 (m, 2H), 363 4.44 (t, J=5.5 Hz, 4H). A yield of 40% was obtained (compared to 45% in literature).

### Polymerization:

Synthesis of pTMC-PEG diblock polymers was carried out as a modification of method described in literature [10]. A range of polymers were synthesized containing 1 kDa or 2 kDa methoxy-PEG block with variable proportions of pTMC (70, 75 and 80% total weight of diblock polymer) by adding a stoichiometric amount of these components to a flask. To ensure dryness of methoxy-PEG and TMC, anhydrous toluene was added and evaporated under reduced pressure, this step was typically performed twice. Reagents were dissolved in anhydrous dichloromethane such that the concentration of TMC was 0.5 M and put under an inert atmosphere. Methanesulfonic acid (MSA) was added dropwise in a (2:1 molar ratio MSA:PEG). The reaction was refluxed for 24 h at 30° C. The mixture was concentrated under reduced pressure, the mixture was then concentrated under reduced pressure and precipitated into excess ice cold diethyl ether. Precipitates were dried, dissolved in 1,4-dioxane and lyophilized. Composition was determined by 1H NMR (500 MHz, CDCl3, δ) 2.05 (q, 1H), 3.4 (s, 377 1 H), 3.65 (s, 2H), 4.25 (t, 2H). Dispersity (Ð) of polymers was determined by gel permeation chromatography (GPC). Yields of 77-93% were achieved (compared to 80-90% in literature).

Simultaneous thermogravimetric analysis and differential scanning calorimetry Polymers were analysed from ambient temperatures to 995° C. for full material characterization and an in-depth characterization over a biologically relevant range.

### Nanoparticle Synthesis

Direct hydration of nanoparticles was achieved by dissolving PEG-pTMC polymer in 50 wt % oligo ethylene glycol (MW 250) at 30° C. The solution was hydrated using Dulbecco's modified phosphate-buffered saline (dPBS) and stirred at 200 rpm for 10 minutes. Following this, the solution was filtered through a 0.2 μm Supor® membrane to remove any large agglomerates. For cisplatin encapsulation into micelles, cisplatin was co-dissolved in the OEG:PEG—pTMC mixture. A 20% (wt/wt %) loading was used. This was stirred to form a homogenous mixture prior to hydration as discussed above.

### Nanoparticle Characterization:

DLS Hydrodynamic diameter (Dh) and PDI were calculated and measured using dynamic light scattering techniques (Malvern NanoSizer and asymmetric field flow field fractionation-multi angle light scattering instrumentation). NPs were assessed with 1H NMR with a 5-second relaxation delay.

### Scanning Electron Microscopy

Nanoparticles were imaged for morphological and size analysis. 5 μL of 10 mg/ml nanoparticles was deposited onto a silicon wafer and left to evaporate overnight in a fume hood. Images taken at Vacc=1 kV with the secondary election detector.

### Cell Models

Cell cultures of OVCAR-3 and SKOV-3 were used as ovarian cancer models and HeLa was chosen as a control model for biocompatibility and cell viability assays. OVCAR-3 were cultured in Roswell Park Memorial Institute (RPMI) 1640 media fortified with 20% foetal bovine serum (FBS), 1% penicillin-streptomycin (PS) and 0.1% human insulin. SKOV-3 were cultured in McCoy's 5a media fortified with 10% FBS and 1% PS. HeLa were cultured in Dulbecco's Modified Eagle Medium (DMEM) fortified with 10% FBS and 1% PS. All cultures were maintained in T75 flasks with 0.2 μm vented filter caps at 37° C. with 5% CO2 and humidity (Incubator, New Brunswick Galaxy 170s). Cultured were passed once flasks reached 70-80% confluency and all tests were conducted using passages lower than 35. During all viability tests cells were seeded into media with stripped FBS.

Internalization and quantification of nanoparticles was characterized using fluorescently labelled nanoparticles, Vybrant DiD™ loaded particles were fabricated (co-dissolution as previously described) followed by spin filtration to remove non-encapsulated dye. 2D Cell cultures were treated with NP(DiD) for 24, 48 and 72 h. 426 Cells were co-stained for nuclei using 16.2 μM Hoescht. Cells were imaged using CLSM or quantified following detachment using CellStream (A2 for DAPI channel, 428 B6 for DiD channel). Corrected total cell fluorescence (CTCF) was calculated.[36] Microtissue penetration was conducted using the same fluorescently labelled NPs. Spheroids (2×103 cells) were seeded 48 h prior to use, 10 spheroids per NP were treated for 24 h with 20 μL stock NP(DiD).[37] Following treatment times spheroids were 432 pooled, stained with 0.5 μM Hoescht for 1 hour to create differential, localized staining then washed with PBS and disaggregated using 500 μL accutase, incubated for 5 minutes at 37° C. to form a single cell suspension. Samples were mechanically pipetted 435 every minute and single cell suspension was validated using light microscopy prior to running on the CellStream (A2 for DAPI channel, B6 for DiD channel). Data was visualized using MATLAB code form CellStream exports to create violin plots of total populations. Efficiency of miRNA uptake was conducted using fluorescently conjugated miRNA (miR34 with a 5′ FAM (fluorescein) modification, Thermo Fisher Scientific; Ser. No. 10/336,022) on cellular monolayers of SKOV-3 wt. Standard direct transfection method was conducted using HiPerFect transfection reagent (Qiagen; #301705) as per manufacturers specifications. Nanoparticle, cell counter staining and imaging all conducted as described above. Uptake thresholding into binary high and low uptake conditions was conducted using quantification histograms and manual identification of population peaks.

### Cell Viability Realtime-Glo™ MT Cell Viability Assay (RT-Glo):

A non-lytic assay that relies on the reducing environment present in the cytoplasm of viable cells allows for successive monitoring of cell population status for up to 96 h post-assay reagent introduction. Cells were seeded in a 96 well plate with white opaque walls (to prevent luminescence interference), seeding densities were 500 (SKOV-3 and 444 HeLa) and 2,000 (OVCAR-3) cells respectively. Cells were allowed to adhere overnight and then treated. PBS and either 5 μM SAHA or 100 nM Doxorubicin were used as positive and negative controls, respectively. Direct transfection of miRNAs was conducted using HiPerFect transfection reagent (Qiagen; #301705) as per manufacturers specifications of the traditional transfection protocol. NanoLuc® enzyme and MT cell viability substrate were diluted in FBS stripped media such that the final concentration was 1:1000 of that provided in the Promega Kit as recommended. Luminescence readings were taking using a FLUOstar Omega microplate reader heated to 37° C. at 0-450, 24-, 48-, 72- and 96 h time points. Viability of microtissues/spheroids was quantified using Celltiter-Glo® 3D as per manufacturers instruction (Promega, G9682), an endpoint assay that contains a potent lytic agent that disrupts spheroid microtissues and then quantifies cell viability based on ATP concentration. ATP is utilized by Ultra-Glo™ rLuciferase enzyme which causes a bioluminescent signal that can be read via spectrophotometer. Prior to use, the Celltiter-Glo reagent was thawed overnight at 4° C. and then left at room temperature for an hour before use. Media volume in each well was adjusted to 100 μL and then 100 μL of reagent added. Microplates were shaken for 5 minutes at 700 rpm and then left to incubate at room temperature for a further 25 minutes to allow the luminescent signal to stabilise prior to reading.

Mycoplasma levels were determined using MycoAlert™ Mycoplasma Detection Kit (Lonza; #LT07-118, UK). MycoAlert Reagent and Substrate were reconstituted in MycoAlert Assay Buffer. NPs were fabricated as previously outlined above, rehydrated to a concentration of 10 mg/mL and stored for 72 h at 4° C. Briefly, 100 μL of sample and 100 μL of MycoAlert Reagent were mixed and incubated for 5 minutes at room temperature. Luminescence was measured using a FLUOstar Omega microplate to provide ‘Reading A’. Following this, 100 μL of MycoAlert Substrate was added and left to incubate at room temperature for a further 10 minutes. Sample luminescence was measured, ‘Reading B’. Ratio of B/A gives final result.

ROS Induction from nanoparticle control treatments was quantified using CM-H2DCFDA ROS probe in 2D. 1×10⁴ cells were seeded in 100 uL per well in a black, opaque 96 well microplate (n=3). Cells were treated for 4, 24 and 48 h with blank nanoparticles. Cells were washed and incubated with 5 μM probe in pre-warmed stripped, phenol-red free media (loading buffer) for 30 minutes at 37° C., ensuring that sample was covered to protect from photobleaching. After incubation the loading buffer was removed, sample was rehydrated in 1×dPBS and fluorescence was monitored using a FLUOstar Omega microplate reader with EM20 filter configuration and gain set to 1000.

Immunogenicity of particles was assessed ex-vivo by monitoring changes in soluble cytokines (list) leukocyte activation and death as previously reported by Radley et al (Radley, G., Pieper, I. L., & Thornton, C. A. (2017). The effect of ventricular assist device-associated biomaterials on human blood leukocytes. https://doi.org/10.1002/jbm.b.33981.

Blood collection: Peripheral blood was collected into 4 mL lithium heparin vacutainer tubes (18 IU lithium heparin salt per mL; Greiner Bio-One, UK). All donated blood was from healthy adult volunteers with informed written consent and under ethics 2020-0035, approved by Swansea University Medical School Research Subcommittee (SUMS RESC).

### Leukocyte Activation and Death

Whole blood cultures were conducted to monitor changes in soluble cytokines. 50 μL of whole blood was added to 150 uL RMPI 1640 fortified with 2 mM 490 GlutaMax β-Mercaptoethanol in a U-bottomed 96-well plate (Greiner Bio-One, UK). Treatments were added directly at this stage and left to incubate for 24 h at 37° C., 5% CO2. Plates were centrifuged at 4° C., 515 g for 7 minutes and supernatant was aspirated and stored at −80° C. until analyzed.

In vivo studies were conducted in female Balb/c nude mice with SKOV-3 Luc lumpectomies xenographs. SKOV-3 Luc cells (1×10⁷ cells 1:1 in Matrigel) were implanted subcutaneously onto the flank of 5 donor female BALB/c nude mice using a 23-gauge needle. When tumours reached approximately 600-800 mm³ the mice were euthanized and the donor tumours resected. Donor tumours were divided into 50 mm³ segments which were then implanted subcutaneously on the right flank of 500 female BALB/c nude mice. When tumours reached approximately 150-200 mm³ the mice were randomly assigned to treatment groups. Mice were imaged pre-dose and at 1, 4, 12, 24 and 48 h post dose of nanoparticles using an In Vivo Imaging System (IVIS). Animals were anaesthetised using a gaseous mix of isofluorane and oxygen, which allowed an optimum plane of anaesthesia throughout imaging. Imaging was performed using a highly sensitive scale and animals were imaged for fluorescence.

A total of 36 female BLAB/c nude mice aged 5-8 weeks were used for the in vivo studies. These animals were purchased from Envigo therefore they required a seven-day acclimatization period. Animals were housed in IVC cages (up to 5 per cage) with individual mice identified by tail mark. All animals were allowed free access to a standard certified commercial diet and sanitised water during the study. The holding room was maintained under standard conditions: 20-24° C., 40-70% humidity and a 12 h light/dark cycle. Each cage was subjected to 51-54 air cages per hour and the holding room had 14 air changes per hour. Tumour cell implantation: SKOV3_luc cells (1×10⁷ cells 1:1 in Matrigel) were implanted subcutaneously onto the flank of 5 donor female BALB/c nude mice using a 23-gauge needle. When tumours reached approximately 600-800 mm³ the mice were euthanised and the donor tumours resected. Donor tumours were divided into 50 mm3 segments which were then implanted subcutaneously on the right flank of female BALB/c nude mice.

When tumours reached approximately 150-200 mm³ the mice were randomly assigned to treatment groups as demonstrated in Table 1.

Animals were imaged weekly for bioluminescence using an In Vivo Imaging System (IVIS). Animals were injected with luciferin (150 mg/kg) 10-15 minutes prior to imaging via intraperitoneal injection. Animals were anaesthetised using a gaseous mix of isofluorane and oxygen, which allowed an optimum plane of anaesthesia throughout imaging. Imaging was performed using a highly sensitive scale. Tumours were measured three times per week using digital callipers. The length, width and depth of the tumour will be measured and used to calculate the tumour volume. The bodyweight of all mice on the study was measured and recorded three times weekly; this information was used to calculate precise dosing for each animal. Mice were observed daily and any signs of distress or changes to general condition e.g. starred fur, lack of movement, difficulty breathing.

Sampling At the end of the study period (day 28) animals were euthanised via carbon dioxide inhalation. Tumours were resected and the whole weight recorded Tumour growth inhibition calculation against vehicle group (T/C) and cisplatin group (T/C′) calculated using equations 1 or 2, respectively.

$$\% \frac{T}{C} Value = \frac{Mean tumour volume on day 28 - Mean starting value}{\begin{matrix}Mean vehicle tumour volume on day 28 - \\ Mean vehicle starting volume\end{matrix}} \times 100 \qquad ( 1 )$$

$$\% {\frac{T}{C}}^{′} Value = \frac{Mean tumour volume on day 28 - Mean starting value}{\begin{matrix}Mean cisplatin tumour volume on day 28 - \\ Mean cisplatin starting volume\end{matrix}} \times 100 \qquad ( 2 )$$

### Results & Discussion

The following demonstrate the development of a novel drug delivery system with an innate affinity for ovarian cancer cell internalisation with the ability to permeate into the core of microtissues based on flow cytometry and drug release to the entirety of the tumour. The presents a unique opportunity to shuttle higher concentrations of cytotoxic chemotherapeutic agents or apply novel synergistic combinations without the concern of patient tolerance and sub optimal doses reaching the disease site.

### Polymer Synthesis and Characterization

By changing fundamental properties of polymers, such as molecular weight and ratio of the hydrophobic to hydrophilic regions, we can customize the downstream nanoparticle that will form from the bulk polymer.

Eight polymers for potential use as drug carriers for ovarian cancer were synthesized using either a 1 kDa or 2 kDa PEG block and various lengths of PTMC, reaction schematic given in FIG. 1.

Validation of the degree of polymerization and thus length of the PTMC repeat was achieved using ¹H NMR, see FIG. 2a, FIG. 3 for NMR spectra example and results of molecular weight calculations are given in Table 2. Additionally, as TMC repeat determination is achieved by comparison of PEG peaks to PTMC peaks in the NMR spectra, an accurate MW was necessary for continued use. MALDI-TOF MS was conducted by the National Mass Spectrometry Facility on PEGs that were not received with this data, an example read out is given in FIG. 3c here the bulk of the material, 95.6% had a MW of 2012. It was also shown to be highly uniform as the majority of the polymer (96.6%) was shown to have a MW between 2006-2012.

Gel permeation chromatography analysis of polymers was conducted to measure polymer dispersity (D). An example of calibration details are given in FIG. 4 and Table 3 gives molecular weights of the polystyrene standards used for calibration.

A condensed over-layered chromatogram for polymers 1-8 is given in FIG. 4d, elution time for all polymers was between 7-11 minutes. Each chromatogram was found to only contain one peak per sample, demonstrating only one polymer entity per sample as shown in FIG. 2b, rather than starting materials and product with varying degrees of polymerizaiton. The uniformity or dispersity of polymers is the main output of interest provided by GPC. Polymers 1,4,5,7,8 were shown to have a dispersity index of ≤1.1, this degree of uniformity is exceptionally narrow and is the required value for analytical standards. Polymers 2, 3 and 6 had dispersities between 1.2-1.3, which still falls within a very narrow molecular weight distribution range, commonly accepted to be 1.2-1.5. Moreover, a narrow molecular weight distribution of polymers is fundamental to produce NPs with narrow PDIs.

Thermal Analysis of polymers, thermal gravimetric analysis (TGA) and differential scanning calorimetry (DSC) was conducted on bulk polymer materials, example for polymer is given in FIG. 2c and thermal analyses for all polymer are given in FIG. 5 and FIG. 6. Due to lack of chilling capabilities, thermal analyses were all conducted above glass transition temperature of polymers. Previous works on similar polymers including PEG45PTMC96 (similar to polymer 4, PEG44PTMC73) have shown glass transition temperatures are below −21° C. [25] Melting points, see Table 2, were determined using DSC output by identification of first spike in endothermic energy (FIG. 6). All polymers were shown to have melting points between 42 and 55° C. Interestingly, this property could be exploited for thermo-stimulated drug release. Speculatively, this could either utilize intrinsic biology cancer and the comparatively increased internal temperature of the tumour or in be used conjunction to tumour thermal ablation. [26-28] Polymer products are collated in Table 2 collated characterization data for polymers 1-8, including NP size, and zeta potential 152 measurements. GPC tailing and Ð given to 1d.p. Table 2. Representative NMR from products of monomer synthesis and polymer synthesis run in CDC₃ are given in FIG. 7 and FIG. 3.

### Nanoparticle Fabrication Using Direct Hydration

Nanoparticles were fabricated from bulk polymers using the direct hydration method as described in Ridolfo et al. and subsequently characterized to understand the morphology, uniformity and charge of nanovectors.[10] Details of NP size and zeta potential are provided in Table 2 and FIG. 8a is the overlay of intensity size distribution of all NPs as determined by DLS (n=3). NP size distribution with SEM is provided in FIG. 9a. Comparisons of fabrication methods and sizing techniques (FIG. 10) shows that direct stirring is favorable to vortex suspension as this leads to a shoulder on the DLS read out. This is likely due to the formation of agglomerates rather than discrete micelles. Additionally, multiple techniques have been used to validate the use of DLS for NP measurement. AF4, an optical technique that differs to DLS by the inclusion of a chromatography system prior to sample reading, showed identical results for NP radii. AF4 can provide more detailed information for samples with multiple distinct NP populations. The results here demonstrate a uniform NP sample, in line with the polymer uniformity as discussed above. The relationship between polymer molecular weights and NP sizes are given in FIG. 9b. FIG. 10c-d gives a comparison of DLS to SEM, a physical characterization technique using NP4 as an example. Z-average obtained from DLS was 168.4 nm (FIG. 10c), average of NPs detected in SEM characterization (FIG. 10d), was 168.6 nm. This reproducibility in size measurements between DLS, AF4 and SEM gives confidence in the use of DLS for further size analysis of these materials.

### Sterility of NP Fabrication Process

GLP considerations are important for potential downstream translation to clinic. As such, following standard NP fabrication techniques, samples were tested for the presence of Mycoplasma using MycoAlert kit. All samples tested were shown to be negative for Mycoplasma contamination. Results for this assay are given in FIG. 11 and Table 4.

### Cytotoxicity and Compatibility

ROS production in vitro from NP exposure to cell lines was conducted using CM-H2DCFDA probe, results given in FIG. 12. ROS production is usually associated with metallic NPs such as iron-oxide, however, material behavior on the nanoscale differs to bulk material making this an area of interest even in the case of polymeric NPs.[29,30] The only NP treatment that gave a statistically significant increase in ROS was 24 h NP6 exposure in SKOV-3 FIG. 12d. All other treatments were well tolerated. There is a clear trend between all treatments and cell lines, 24 h treatments seems to be consistently higher than the other time points. This timeline matches the peak of NP internalization/uptake and the ROS levels could be a result of an overwhelming influx of NP. After this timepoint, all cell lines show decreases in ROS, indicative of recovery. Another consideration is that following 24 h from initial exposure, antioxidant genes such as thioredoxin or peroxiredoxin have been upregulated thus the cell is reducing ROS levels.[31] Regardless of the molecular machinery, the NPs are well tolerated by the cells without leading to a dramatic increase in ROS.

Biocompatibility of NPs in vitro A large range of concentrations were assessed (0.2-200 μM) in both OVCAR-3 and SKOV-3 using cell viability assay Realtime-Glo™. The full results of which are given in FIG. 13 (OVCAR-3) and FIG. 14 (SKOV-3), In 20 μM treatments (FIG. 12a-b) all NPs were well tolerated or the viability recovers by the 48 h timepoint onwards, as seen with NPs 4 (OVCAR-3 only), 6 (both cell lines) and 8 (both cell lines). As Realtime-Glo uses cellular metabolic levels, when samples appear to ‘recover’ as seen here, it is plausible that these results are in response to a high energetic demand rather than compromised cell integrity. This timeline matches the peak internalization point for NP uptake, which would account for this drop in energy levels as endocytosis is known for its high energetic demand.[32] However, this is not the case for NP5, in OVCAR-3 (FIG. 12a) viability decreased to 58% at 72 h (p s 0.0001) and in SKOV-3 FIG. 12b) viability decreased to 54% at 72 h (p s 214 0.0001). This indicates that NP5, would not be a good candidate for treatments if used at this concentration, FIG. 13e and FIG. 14e show that this particle does not have this effect at 10 μM or lower, where viability at 72 h is 94.5% (OVCAR-3, ns) and 136.7% (SKOV-3, ns). Interestingly, whist NP6 treatments caused an increase in ROS, this isn't reflected in the viability assessments. Components used in polymer NPs i.e. PEG 2000, trimethylene carbonate (TMC) and oligo-ethylene glycol—250 (OEG) were tested for biocompatibility, see FIG. 15. As micelles are subject to degradation through via kidneys, liver or lysosome, it is important to also consider the possibility of cell exposure to polymer fragments. [33,34] None of these materials were shown to significantly decrease viability, the greatest decrease in viability was 2% following 200 μM treatment of PEG 2000 (OVCAR-3 FIG. 15a; ns).

Compatibility analysis of NPs in 3D was conducted after 24, 48 and 72 h exposure to NPs results of 25 μM treatments in OVCAR-3 and SKOV-3 are given in FIG. 12.e-f, additional results in HeLa are provided in FIG. 22. In OVCAR-3 spheroids at 24 h treatments both NP1 and NP3 231 caused a decrease in viability, by 31.4% (p<0.05) and 51.4% (p<0.001), respectively. NP treatments of 48 and 72 h did not decrease viability. In accordance to OVCAR-3 233 treatments, SKOV-3 spheroids also showed a decrease in viability at 24 h with NP1 and NP3 treatments by 45.6% (ns), and 48.8% (ns). No other treatments in SKOV-3 spheroids lead to a decrease in viability. Reinforcing this trend, 24 h treatments of NP1 in HeLa spheroids decreased viability by 34.1 (ns) FIG. 16a. There is a consistent increase in fold viability at 48 and 72 h following NP treatment across all three cell lines.

Internalization/uptake of NPs in 2D was conducted over several timepoints, below in FIGS. 17 and 19 images of 24 h treatments are given for NP4, NP7 and NP8. At this timepoint the internalization levels of NP8 in OVCAR-3 (FIG. 19.a and c) are significantly higher (10⁷ A.U.) than those seen in the other NP treatments (~106; p<0.0001), internalization rates level out at 48 h (10-15⁵ A.U.) and at 72 h NP4 (12⁶ A.U.) and NP7 (11⁶ A.U.) are higher than NP8 (11⁵ A.U.; p<0.001 and p<0.01 respectively), indicating cellular degradation or metabolism over the timeframe investigated. In SKOV-3, all 3 NPs appear to have similar internalization rates at 24 h, however, reviewing the stability of NPs across the timepoints investigated (FIG. 17a-b), highlights that NP4 and NP8 have consistently high fluorescence levels as opposed to NP7 which declines rapidly from 24 h. In SKOV-3 (FIG. 19.b-c), quantified levels of uptake show that NP4, NP7 and NP8 have higher uptake rates than other NPs (FIG. 17)

Endocytosis elucidation was conducted on the NPs with the highest quantified uptake: NP4, NP7 and NP8. Universally, there is not one single route of endocytosis that was solely responsible for all NP internalization and the endocytosis pathway attributed to the highest proportion of uptake was Arf6 (FIG. 17.e-f), a key mediator of membrane and endocytic recycling—protein expression heatmap for selected HGSOC and healthy ovarian cell lines (FIG. 17g) shows ubiquitously high expression across phenotypes.[35] AfterArf6, the most prevalent route of endocytosis of the NPs in OVCAR-3 WT was macropinocytosis (FIG. 17e), and Dynamin-dependent in SKOV-3 WT, except for NP8 (FIG. 17f). Interestingly whilst there is a clear trend, the exact breakdown of endocytosis pathways is not the same for all particles in each cell line, nor for a single NP across both cell lines. In some instances, chemically inhibiting some routes of endocytosis actually resulted in a higher overall uptake of NPs, indicative of a compensatory cellular response, (FIG. 17d,f) where treatment of SKOV-3 WT with chloroquine (an inhibitor of Clathrin-mediated endocytosis) lead to a minor increase (3%) in cell population positive for NPs and a significant increase in mean florescence of 3×10⁵ (p<0.001; FIG. 17d). Additionally, whilst few inhibitors lead to a reduction in the proportions of cells positive for NPs, some lead to a drastic decrease in fluorescence per cell (FIG. 17c-f). This phenomenon is seen with NAV 2729, an inhibitor of caveolae-mediated endocytosis where complete inhibition isn't achieved but a significant drop in fluorescence by 90% in both OVCAR-3 WT and SKOV-273 3 WT (p<0.001) evidence of partial inhibition.

Penetration/uptake of NPs in 3D NP penetration/uptake in spheroids/microtissues was analyzed after 24 h exposure, preformed spheroids were dosed with 20 μL of stock NPs (0.2 mg; 6.8-13.2×1010 NPs) loaded with Vybrant DiD stain. Spheroids were stained with Hoechst and then disaggregated using accutase and mechanical pipetting. The single cell suspension was ran on the CellStream and fluorescence measured. Outputs are visualized below in FIG. 18. Generally, although there is complete penetration of the spheroid there is also a clear difference in the degree of internalization between OVCAR-3 and SKOV-3 spheroids. OVCAR-3 (FIG. 18a) shows consistently lower uptake than SKOV-3 (FIG. 18b). General trend for NP uptake is consistent across OVCAR-3 and SKOV-3 cell lines with NPs 4,7 and 8 having higher levels of internalization than other NPs. This is consistent with HeLa uptake FIG. 16b. Interquartile values are given in Table 5 below. There is a clear difference in uptake patterns between OVCAR-3 WT and SKOV-3 WT, in the latter all interquartile ranges and mean fluorescence are similar (means: 1.8-2.0E⁵). Whereas there is a major difference in uptake between the particles in OVCAR-3 WT after 24 h exposure times, ranging from 1.8E³-2.2E⁵, a trend also seen in the 2D uptake work (FIG. 19c).

IC50s were compared for Free Drug Cisplatin and nanoparticle encapsulated Cisplatin in 2D cultures using WT IC50s in OC cell lines (FIG. 21). Surprisingly, encapsulated cisplatin was able to reinstate WT sensitivity in SKOV-3cis monolayers with a stable response over the period investigated.

The efficacy of Nanoparticle encapsulated combination therapies was also compared with analogous, non-encapsulated combination therapies, revealing potential utility for the treatment of refractory cancers. Specially, the efficacy of co-encapsulated HDACi2 with cisplatin or gefitinib was assessed using cell line models of low-grade serous OC (LGSOC; SKOV-3 wt) and HGSOC (OCCAR-3 wt) and their cisplatin resistant variants (SKOV-3cis and OVCAR-3cis) as models of secondary or refractory cancers.

The contrasting effects of HDACi2-co-adminstation with cisplatin (“HC”) versus a co-encapsulated HDACi2—cisplatin nanoparticle formulation (“NP(HC)”) are shown in FIG. 25. In SKOV-3 wt, HC treatments were found to reduce viability by 36.8±3.0% after 24 h (P<0.0001), 44.2±5.6% after 48 h (P<0.0001) and 58.4±5.2% after 72 h (P<0.001). However, co-encapsulation was found to further enhance cytotoxicity of this combination, with NP(HC) reducing viability by 82.2±1.3% after 24 h, 75.0±3.0% after 48 h, and 71.9±2.6% (P<0.0001 against negative control for all timepoints, p<0.0001 or p<0.05 against positive control and HC treatments). In OVCAR-3 wt (FIG. 5b) HC treatment reduced viability by 63.1±1.6% after 24 h, 88.6.±1.3% after 48 h and 86.6±2.3% after 72 h (p<0.0001 for all time points) and NP(HC) enhanced efficacy initially with viability reduced by 64.8±4.8% after 24 h, however, NP)HC) was less effective than HC at 48 and 72 h where viability was reduced by 81.4.±1.6% and 61.6±4.5%, respectively, (p<0.0001 for all timepoints). HC treatments produced similar cytotoxicity in SKOV-3cis (FIG. 25c) as that observed in SKOV-3-wt, with viability reductions of 30.4±2.6%, 46.2±4.0% and 58.7±3.8% across the timepoints. (p<0.0001). NP(HC) further reduced viability by 68.9±2.8% at 24 h, 78.2±1.4% after 48 h and 79.8±2.9% after 72 h (p<0.0001 against negative control at all timepoints and positive control at 24 h and 48 h). Comparing HC to NP(HC), encapsulation was found to significantly enhance cytotoxicity across all time points (p<0.0001 or p<0.01).

OVCAR-3cis was found to be more resistant to HC treatments that all other cell lines investigated. HC treatments were found to reduce viability by 39.2±4.1% after 24 h (p<0.0001 to both controls) and 42.5±3.2% after 48 h (p<0.0001 to both controls), however, by 72 h cells made a significant recovery to 92% (ns). NP(HC) was far more effective across the investigated period with viability reduced by 77.7±2.1%, 73.5±1.7% and 57.2±3.4% (p<0.0001 against all controls and HC treatment). This observed difference in cytotoxicity between free and encapsulated API (49.2% increase in cytotoxicity with NP(HC) versus HC) was the greatest observed enhancement observed throughout the study. In addition, the API combination produced the greatest cytotoxic effect in HeLa (a cervical cancer line) out of all the combinations tested, viability was reduced by 54.9±8.0% and 62.3±5.5% for HC and NP(HC) after 72 h exposure.

### Immunogenicity

Tolerance and biodistribution studies were conducted of NP4, NP7 and NP8. Mouse body weight (FIG. 20a), a general marker of health demonstrated that even at a very high concentration (140 mg/kg), the material was well tolerated and showed a favorable accumulation in the tumor without a targeting moiety and with intravenous administration (systemic exposure; FIG. 20b-d). Throughout the 3 day biodistribution study, there is a clear accumulation trend in the tumor (FIG. 20c) with final levels being over 900% (p<0.01; normalized to pre-dose levels) for both NP4 and NP7 and 1200% (p<0.001) for NP8. This is further validated by the ex vivo imaging (FIG. 20b,d) that showed a slightly different fluorescence distribution of over 1600% (p<0.05; 304 normalized to ex vivo vehicle control tumors) for both NP4 and NP8, and a slightly lower value of ~1200% in NP7.

### Nano-Enabled Gene Therapies

Traditional methodologies of (mi)RNA transfection are limited. HiPerFect, a well-established transfection reagent was investigated for internalisation potential (FIG. 24, panel one: “Direct transfection”). The fluorescence imaging of miR34 (grey LUT) following traditional transfection showed a non-uniform uptake pattern of the material into cells. Signal was observed to be predominantly cytoplasmic. Conversely, imaging of the nanoparticle (red LUT) counterpart (FIG. 24, panel two: “NP”) showed a more uniform population uptake pattern with more distinct speckling with both cytoplasmic and nuclear fate. Quantified uptake of both treatments (FIG. 24a) highlights the overall positive cells for signal was higher in NP treatment (95±4%) compared to transfection reagent (61±18%; p<0.0001). Following classification of cells with high and low signal, it was observed that NP-treated was generally a high signal (~80%) and traditional transfection resulted in more low fluorescence (~40%) than high (~20%). Therapeutic potential of a pro-apoptotic miRNA following traditional transfection and nano-enabled (FIG. 24b-c) at discrete doses (60 nM and 40 nM) demonstrated that nanoparticle encapsulation can enhance and expedite therapeutic effect of RNA. The latter was clearly observed in OVCAR-3 wt (FIG. 24c) where at 24 h traditional transfection did not lead to a reduction in cellular viability at either dose, whereas nanoparticle variant had reduced viability by over 70% with both doses (p<0.0001). In SKOV-3 wt (FIG. 24b), NP(miRNA) also produced an expedited response with reductions of between 70-75% for 40 and 60 nM respectively. Following 72 h, effect of 60 nM direct transfection were akin to those achieved following 24 h of NP(miRNA), 71.4% and 70.0%, respectively. In OVCAR-3 wt, the enhancement in therapeutic effect of RNA was clear. The maximal reduction in cellular viability following direct transfection was 52.0±9.7% (p<0.01; FIG. 24c) with 60 nM after 72 h compared to 80.5±1% (p<0.0001) with matched NP(miRNA).

### SUMMARY

We have shown a methodology for the preparation of novel biodegradable, drug loaded nanoscale vectors and established their utility using cisplatin loaded vectors in the treatment of ovarian Cancer. Direct hydration provides a uniquely facile method for the preparation of well-defined, drug-loaded PEG-PTMC vesicles that can be prepared with physical features (such as size) dictated by their chemical composition. The formulation of PEG-PTMC into discrete, nanoparticles is essential for its utilisation as an effective drug delivery vector. There are many copolymers and formulation protocols that do not result in the formation of discrete, well-defined particles and that, moreover, require the use of toxic organic solvents.

In vitro cytotoxicity of cisplatin-loaded vectors towards ovarian tumour cells, with free cisplatin as a reference, yielded excellent efficacy following intraperitoneal and, moreover, intravenous administration, which points towards a therapeutic improvement in using the present delivery mode and formulation. Moreover, this platform can readily be applied to a wide range of drugs and to engineering more complex copolymeric nanosystems for application in cancer, and in particular solid tumour, therapy; free from the constraints of complex fabrication processes, background toxicity or impeded drug efficacy.

TABLE 1

| Group | n | Treatment | Dose | Dosing route |
|---|---|---|---|---|
| 1 | 6 | Vehicle | n/a | IV |
| 2 | 6 | Cisplatin | 5 mg/kg | IV |
| 3 | 6 | NP(CisPt) | 5 mg/kg | IV |
| 4 | 6 | NP(CisPt) | 5 mg/kg | IP |

TABLE 2

| | | | | | | | | | DLS | | |
|---|---|---|---|---|---|---|---|---|---|---|---|
| | | NMR | | | GPC | | | DSC | | | [symbol] |
| | [symbol] | [symbol] | [symbol] | [symbol] | [symbol] | | | [symbol] | [symbol] | | [symbol] |
| [symbol] | [symbol] | [symbol] | [symbol] | [symbol] | [symbol] | [symbol] | [symbol] | [symbol] | [symbol] | [symbol] | [symbol] |
| 1 | [symbol] | [symbol] | [symbol] | [symbol] | [symbol] | [symbol] | [symbol] | [symbol] | [symbol] | [symbol] | [symbol] |
| 2 | [symbol] | [symbol] | [symbol] | [symbol] | [symbol] | [symbol] | [symbol] | [symbol] | [symbol] | [symbol] | [symbol] |
| 3 | [symbol] | [symbol] | [symbol] | [symbol] | [symbol] | [symbol] | [symbol] | [symbol] | [symbol] | [symbol] | [symbol] |
| 4 | [symbol] | [symbol] | [symbol] | [symbol] | [symbol] | [symbol] | [symbol] | [symbol] | [symbol] | [symbol] | [symbol] |
| 5 | [symbol] | [symbol] | [symbol] | [symbol] | [symbol] | [symbol] | [symbol] | [symbol] | [symbol] | [symbol] | [symbol] |
| 6 | [symbol] | [symbol] | [symbol] | [symbol] | [symbol] | [symbol] | [symbol] | [symbol] | [symbol] | [symbol] | [symbol] |
| 7* | [symbol] | [symbol] | [symbol] | [symbol] | [symbol] | [symbol] | [symbol] | [symbol] | [symbol] | [symbol] | [symbol] |
| 8* | [symbol] | [symbol] | [symbol] | [symbol] | [symbol] | [symbol] | [symbol] | [symbol] | [symbol] | [symbol] | [symbol] |

[symbol]

indicates data missing or illegible when filed

TABLE 3

Calibration standard

| | 1 | 2 | 3 | 4 | 6 | 7 |
|---|---|---|---|---|---|---|
| MW (kDa) | 2.1 | 5.6 | 9 | 18 | 33 | 56 |

TABLE 5

| | | |
|---|---|---|
| OVCAR-3 WT | SKOV-3 WT | HeLa |

| | [symbol] | | [symbol] | | [symbol] | |
|---|---|---|---|---|---|---|
| NP | [symbol] | [symbol] | [symbol] | [symbol] | [symbol] | [symbol] |
| NP [symbol] | [symbol] | [symbol] | [symbol] | [symbol] | [symbol] | [symbol] |
| NP [symbol] | [symbol] | [symbol] | [symbol] | [symbol] | [symbol] | [symbol] |
| NP [symbol] | [symbol] | [symbol] | [symbol] | [symbol] | [symbol] | [symbol] |

[symbol]

indicates data missing or illegible when filed

TABLE 4

| Luminescence Ratio | Interpretation |
|---|---|
| <0.9 | Negative reading |
| 0.9-1.2 | Borderline - retest |
| >1.2 | Mycoplasma contamination |

1.-24. (canceled)

25. A nanoparticle for encapsulation of one or more active pharmaceutical ingredient, said nanoparticle comprising an amphiphilic block copolymer, wherein said amphiphilic block copolymer comprises:
i. a hydrophobic polymer block having a weight average molecular weight (M_{w}) ranging from about 4 kDa to about 10 kDa, and comprising structural units of trimethylene carbonate; and
ii. a hydrophilic polymer block having a M_{w} ranging from about 1.5 kDa to about 2.5 kDa, and comprising structural units of one or more alkylene glycols,
wherein said nanoparticles have an average particle size, measured according to dynamic light scattering, from about 85 to 150 nm.

26. The nanoparticle according to claim 25, wherein said amphiphilic block copolymer is a diblock copolymer.

27. The nanoparticle according to claim 25, wherein said hydrophilic polymer block comprises structural units of one or more alkylene glycols selected from ethylene glycol, propylene glycol or mixtures thereof.

28. The nanoparticle according to claim 27, wherein said hydrophilic polymer block comprises structural units of ethylene glycol.

29. The nanoparticle according to claim 25, wherein said amphiphilic block copolymer comprises from about 15% to about 60% of said hydrophilic polymer block, based on the M_{w} of the amphiphilic block copolymer.

30. A drug delivery vector according to claim 25, comprising one or more active pharmaceutical ingredients encapsulated within the nanoparticle.

31. The drug delivery vector according to claim 30, wherein said one or more active pharmaceutical ingredient is an anti-cancer therapeutic, preferably an alkylating agent such as cisplatin.

32. The drug delivery vector according to claim 30, wherein said one or more active pharmaceutical ingredients are selected from small molecule drugs, biopharmaceuticals and combinations thereof.

33. The drug delivery vector according to claim 30, comprising a combination of at least two co-encapsulated APIs, preferably wherein each API has a different therapeutic mode of action.

34. The drug delivery vector according to claim 33, wherein each of said APIs is an anti-cancer agent, and wherein said anti-cancer agents are selected from epigenetic modifier, an alkylating agent, and a mitotic inhibitor.

35. The drug delivery vector according to claim 34, comprises at least two, and optionally all three, co-encapsulated anti-cancer agents selected from an epigenetic modifier, an alkylating agent, and a mitotic inhibitor.

36. The drug delivery vector according to claim 30, wherein the weight ratio of said one or more active pharmaceutical ingredients: amphiphilic block copolymer is less than 1:3, and preferably less than or equal to 1:4.

37. A pharmaceutical or veterinary composition according to claim 30, comprising the drug delivery vector and a pharmaceutical or veterinarily acceptable diluent carrier and/or excipient.

38. A process for preparing the nanoparticle according to claim 25, the process comprising:
i. dissolving said amphiphilic block copolymer in an oligomeric alkylene glycol solution to prepare an alkylene glycol-polymer solution; and
ii. hydrating said alkylene glycol-polymer solution by addition of water, an aqueous solution or an aqueous buffer to prepare said nanoparticle.

39. A process for preparing the drug delivery vector according to claim 30 by encapsulating one or more active pharmaceutical ingredients within a nanoparticle, wherein said process comprises:
i. co-dissolving said amphiphilic block copolymer and said one or more active pharmaceutical ingredients in an oligomeric alkylene glycol solution to prepare an alkylene glycol-polymer solution; and
ii. hydrating said alkylene glycol-polymer solution by the addition of water, an aqueous solution or an aqueous buffer to prepare said drug delivery vector.

40. The process according to claim 39, wherein the weight ratio of said one or more active pharmaceutical ingredient: amphiphilic block copolymer is less than 1:3, preferably less than or equal to 1:4.

41. The process according to 38, wherein said oligomeric alkylene glycol comprises from 2 to 10, preferably from 4 to 6, repeat units derived from ethylene glycol, propylene glycol or mixtures thereof.

42. The process according to claim 41, wherein said oligomeric alkylene glycol is an oligomeric ethylene glycol according to Formula (I): 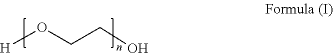
wherein n is an integer between 2 and 10, preferably an integer between 4 and 6.

43. The process according to claim 39, wherein said active pharmaceutical ingredient is an anti-cancer therapeutic, preferably an alkylating agent such as cisplatin.

44. A drug delivery vector according to claim 30, or a pharmaceutical or veterinary composition, for use as a medicament, preferably for use in the treatment of cancer.

45. A drug delivery vector according to claim 30, or a pharmaceutical or veterinary composition, for use in manufacture of a medicament to treat cancer.

46. The drug delivery vector or the pharmaceutical or veterinary composition according to claim 44 wherein said cancer is a solid tumour cancer, preferably ovarian cancer.

47. A method of treatment comprising administering to a subject in need thereof an effective amount of a drug delivery vector according to claim 30 or a pharmaceutical or veterinary composition.

48. The method according to claim 47, wherein said subject has cancer, preferably a solid tumour cancer, and still more preferably ovarian cancer, and wherein the drug delivery vector comprises an anti-cancer therapeutic, preferably an alkylating agent such as cisplatin.
